(19) United States
(12) Patent Application Publication
(10) Pub. No.: US 2026/0256539 A1
(43) Pub. Date: Sep. 3, 2026

(54) Filter Assembly And Surgical Smoke Evacuation Unit

(71) Applicant: Stryker Corporation, Portage, MI (US)

(72) Inventors: Matthew Smith, Parchment, MI (US); Michael Zollinger, Chelsea, MI (US); Michael James Irvine, Byron Center, MI (US); Troy E. Durnell, Lakewood, NY (US); Jordan Lada, Kalamazoo, MI (US); Rawley Stanhope, Middleville, MI (US); Grant Westphal, Delton, MI (US); Brandon Lee Laure, Satellite Beach, FL (US); Anthony J. Sall, Zeeland, MI (US)

(73) Assignee: Stryker Corporation, Portage, MI (US)

(21) Appl. No.: 19/491,217

(22) PCT Filed: Sep. 20, 2024
(86) PCT No.: PCT/US2024/047737

§ 371 Date: Dec. 8, 2025

Related U.S. Application Data
(60) Provisional application No. 63/584,593, filed on Sep. 22, 2023.
(60) Provisional application No. 63/646,136, filed on May 13, 2024.

(51) Int. Cl. A61B 90/00; A61B 18/00; A61B 18/14

(52) U.S. Cl. CPC A61B 90/08; A61B 18/14; A61B 2018/00178; A61B 2218/008; A61B 2562/16; A61B 2562/182

(57) ABSTRACT

A filter assembly for a smoke evacuation unit. A sensor is positioned within a housing of the filter assembly. The sensor may be a capacitive sensor disposed near one or more inlet ports. A controller may operate a vacuum source based on the sensor detecting operation of a powered surgical device, for example, though changes in electric field detected through the inlet ports. The filter assembly may include a shielding cover providing an electromagnetic barrier. The shielding cover may be in electrical communication with a contact pad configured to engage a terminal of the smoke evacuation unit. The contact pad may be disposed within a recess on a side of the housing. Electrical contact between the contact pad and the terminal may ground the shielding cover, and the controller may authenticate the filter assembly based thereon. A liquid collection compartment may be disposed beneath a filter compartment within the housing.

### PRIORITY CLAIM

This application claims priority to and all the benefits of United States Provisional Patent Application No. 63/646,136, filed on May 13, 2024, and United States Provisional Patent Application No. 63/584,593, filed on Sep. 22, 2023, the entire contents of each being hereby incorporated by reference.

### BACKGROUND

Surgical smoke includes aerosolized combustion byproducts produced by heat-generating surgical instruments, such as lasers, electrosurgery, ultrasonic devices, drills, and saws. The smoke produces vapors of aerosolized chemicals and substances that can be hazardous to health, including carcinogenic matter, blood and tissue particles, bacteria, and viruses. Literature suggests that perioperative teams exposed to surgical smoke report twice as many respiratory health issues as the general public. In certain instances, the smoke can be thick enough to obscure vision, especially during longer operations where cauterizing tools are heavily used.

Recent efforts have been directed to reducing and eliminating the presence of surgical smoke in the operating room. A smoke evacuation unit may include a vacuum source, and a replaceable filter assembly including filter media may be removably arranged between the suction instrument and the vacuum source. The smoke evacuation unit may be implemented as a console situated on a tabletop within the operating room. Other implementations include the smoke evacuation unit being structurally and functionally integrated with a medical waste management system, for example, the Neptune system manufactured by Stryker Corporation (Kalamazoo, Mich.) and disclosed in commonly-owned United States Patent Publication No. 2007/0135779, published Jun. 14, 2007, the entire contents of which are hereby incorporated by reference. The system disclosed therein includes a controller configured to adjust the speed of the vacuum source based on particulate passing through the filter assembly being detected by an optical sensor. While the optical sensor has provided beneficial responsiveness to the smoke evacuation unit, it generally requires the vacuum source be operating in a near-continuous or continuous manner to detect the particulate being drawn through the filter assembly until the particulate limit is exceeded. Therefore, there is a need in the art for a system that activates the vacuum source based on energizing of a powered surgical device (e.g., the electrosurgical tool) cither as an alternative or in addition to the optical sensor—to improve system responsiveness and permit the vacuum source to be deactivated until needed, thereby reducing noise within the operating theatre. It is further desirable to do so in a manner that is unsusceptible from electromagnetic (EM) noise within the operating room, for example, due to various powered medical devices being activated nearby. Existing solutions include external clips that are susceptible to the EM noise and are cumbersome to set up prior to commencement of the procedure.

The filter assembly should be replaced after its operational life. With the filter assembly being a replaceable component, non-genuine articles often permeate medical facilities. These non-genuine articles are inferior to those manufactured by original equipment manufacturers (OEMs). For example, the non-genuine articles may use lesser quality filtration materials, and/or fail to provide for the aformentioned EM-based activation, resulting in inferior safety for the surgical personnel in the operating room. Therefore, there is a further need in the art for a filter assembly that provides means for ensuring its authenticity and compatibility with the smoke evacuation unit, and done so in a manner that is not prohibitively expensive to be implemented on a disposable component. Additional shortcomings in the art overcome by further inventive aspects will be readily appreciated from the disclosure.

### SUMMARY

Aspects of the present disclosure are directed to a filter assembly for a smoke evacuation unit. The filter assembly includes a sensor disposed within the housing and configured to detect activation, deactivation, and/or changes in power supplied to a powered surgical device, for example, an electrosurgical instrument. The sensor may be a capacitive sensor configured to detect changes in electric field caused by changes in alternating current being supplied through a power cord of the powered surgical device. The changes in the electric field may be detectable through one or more inlet ports of the filter assembly and to which one or more suction tubes are removably coupled.

The filter assembly includes a housing configured to be removably inserted or disposed within a filter receptacle of the smoke evacuation unit. An electronic connector is disposed on a rear side of the filter assembly and configured to provide electronic communication with an electronic connector of the smoke evacuation unit. The housing may be formed from a cover coupled to a filter casing. In certain implementations, the cover may be a shielding cover configured to provide an EM barrier to limit, dampen, reduce, or prevent changes in electric field therethrough. The EM barrier may be caused by conductive material being coupled to, formed with, or disposed within the shielding cover. The conductive material may cover most of a front of the filter assembly, and in particular, nearly an entirety of the portion of the filter assembly that is exposed from the filter receptacle of the smoke evacuation unit. The filter receptacle of the smoke evacuation unit may also be shielded, and therefore the changes in the electric field may generally be detectable by the sensor only through the inlet ports. In certain implementations, the port covers may be at least partially formed from conductive material(s) configured to limit, dampen, reduce, or prevent changes in electric field therethrough. The port covers are openable to couple suction tubes to the inlet ports, thereby providing one or more paths through which the changes in electric field may propagated to within the housing to be detected by the sensor.

The shielding cover may include a casing, and a shielding layer coupled to an inner surface or outer surface of the casing. An exemplary implementation of the shielding cover includes an inner casing, an outer casing, and the shielding layer disposed between the inner casing and the outer casing. The shielding layer is at least partially formed from conductive material. The shielding layer may be contoured to a profile of the inner casing other than the port panel. Alternative variants include the shielding cover formed from a polymeric material with the conductive material embedded or impregnated therein, or a monolithic cover of conductive material such as metal.

The sensor is disposed within the housing. The sensor may be coupled to the shielding cover, for example, positioned adjacent to a port panel defining the inlet ports. In particular, the sensor may be disposed on an inner side of the port panel and in a sensor compartment defined by the inner casing. The sensor compartment may be further defined by a partition spaced apart from the port panel. The sensor compartment may include at least one coupling feature configured to support the sensor therein. The inlet ports bypass the sensor compartment to open into a smoke ingress compartment of the filter assembly.

The sensor may include an upper portion disposed above, below, and/or between one or more of the inlet ports. The sensor may be spaced apart from the conductive material of the shielding cover by a minimum distance so as to limit potential destruction of the electric field as being sensed by the sensor. For example, the sensor may not extend above an uppermost inlet port. Alternatively, the sensor may be two sensors positioned between respective pairs of the inlet ports. The sensor configured to detect the changes in the electric field may be a capacitive sensor, but other types of sensors configured to wirelessly detect changes in energy characteristics through the inlet ports are within the scope of the present disclosure.

The EM barrier may be further facilitated by the shielding cover being grounded to protective earth, for example, through a contact pad engaging a grounding unit of the filter receptacle. The shielding layer of the shielding cover is in electrical communication with the contact pad. A conductive bridge may be positioned to directly contact a conductive flange of the shielding layer. The conductive bridge may include at least one bend to traverse an edge geometry of the inner casing such that the conductive bridge extends along the side of the housing. The opposite end of the conductive bridge may also include a bend shaped to cause resilient contact with an inner surface of the contact pad. Alternatively, a soldered wire lead or another means of electrical communication may be provided. With the filter assembly fully inserted within receptacle, an electrical pathway is established from the shielding layer, through the conductive bridge, the contact pad, the terminals, and to protective earth.

One of the sides of the housing may include a step. The step defines a recess extending distally or forward from the rear side to a position distal to the contact pad. The size and shape of the recess may be complementary to the size and shape of a casing of the grounding unit of the filter receptacle. The step may define an upper portion of the side that itself defines one or more windows. The contact pad is disposed within the housing and secured to an inner surface of the housing for at least a portion of the contact pad to be exposed through the windows. The windows are sized and positioned such that, with the filter assembly fully inserted into the receptacle, the terminals of the grounding unit directly contact the contact pad. The contact between the conductive components establishes the electrical pathway, and current or other signals may be transmitted from the contact pad to the terminals.

The geometry of the step may result in zero lines of symmetry of the rear side of the housing such that the filter assembly is required to be inserted into the filter receptacle in a single orientation. Further, the housing may include a ridge configured to be engaged by a complementary one of the terminals of the receptacle to provide defeatable retention of the filter assembly within the receptacle. The ridge may define a portion of at least one of the windows. The ridge may be oriented vertically and extend between the step and the upper side of the housing. The shielding cover may also be shaped to define a cavity sized to provide the handle to a user for manipulating the filter assembly, such as installing into or removing from the receptacle, or otherwise carrying it about the medical facility. The handle may be positioned opposite the port panel.

The filter casing of the filter assembly defines a filter media compartment, and a filter may be disposed within the filter media compartment. The filter may be a filter stack formed from layers of filter media of varying characteristics. In certain implementations, a liquid collection compartment may be disposed beneath the filter media compartment. A partition may separate the liquid collection compartment from the filter media compartment. The position and orientation of the partition provides for the liquid collection compartment formed along the length of the housing. A sorbent may be disposed within the liquid collection compartment to limit or eliminate sloshing of liquid therein. The sorbent may include an antimicrobial agent to limit or prevent odors, and/or a reagent configured to change color when saturated with liquid.

In certain implementations, the filter assembly includes a liquid separator configured to separate liquid entrained within the surgical smoke prior to the fluid encountering the filter. The barrier is further sized and shaped to direct gas of the surgical smoke along a tortuous path, wherein momentum of the fluid about a lower edge of the barrier causes the liquid to be removed from the gas. The liquid descends into the liquid collection compartment, whereas the gas is drawn into the filter.

Based on activation, deactivation, or adjustment of the power supplied to the electrosurgical instrument or other powered surgical device, the sensor is configured to detect the changes in the electric field through the inlet ports, and generate signals therefrom. A second sensor, for example, an optical sensor, may be configured to detect particulates associated with the surgical smoke, and generate signals therefrom. The signals from one or both of the sensors are transmitted to and through a printed circuit board (PCB) unit including the electronic connector, and further to the controller of the smoke evacuation unit. The controller is configured to operate the vacuum source based on determinations from the received signals.

In view thereof, the smoke evacuation unit may be operated in any number of predetermined or configurable modes. In an instrument operation mode, the vacuum source initially off or at a lower level. Based on the signals received from the sensor indicative of a change in power delivered to the electrosurgical instrument, the controller operates the vacuum source to start, increase, decrease, or stop the level of suction. For example, in response to the electrosurgical instrument being activated, the vacuum level is increased. The controller may analyze the received signals against a predetermined limit and/or according to a predefined algorithm. The instrument operation mode may also utilize the data from both of the sensors. For example, the controller decreases the vacuum level to a non-zero level upon a detected deactivation of the electrosurgical instrument, and permit the vacuum source to continue operating at the lower level until particulates detected by the second sensor no longer exceeds the predetermined threshold, or for a predetermined period.

Therefore, a first inventive aspect of the present disclosure is directed to the filter assembly for the smoke evacuation unit. The housing defines a suction outlet, and one or more inlet ports configured to receive a suction tube. The filter is disposed within the housing between the one or more inlet ports and the suction outlet. A sensor is disposed within the housing and configured to detect changes in electric field through the one or more inlet ports, which is indicative of changes in power supplied to a powered surgical device. The powered surgical device may be an electrosurgical instrument.

In certain implementations, the housing includes the port panel defining the one or more inlet ports. The sensor is disposed adjacent to the inner side of the port panel. The partition may be spaced apart from the inner side of the port panel to define the sensor compartment therebetween. The sensor is disposed within the sensor compartment. One or more coupling features may be disposed within the sensor compartment and configured to support the sensor. The inlet ports may extend through the partition to open into the smoke ingress compartment.

In certain implementations, the capacitive sensor includes a curvilinear edge contoured to at least one of the inlet ports. The inlet ports may include an upper inlet port, a middle inlet port, and a lower inlet port. The capacitive sensor may include an upper portion disposed between the upper and middle inlet ports, and a lower portion disposed between the middle and lower inlet ports. The upper and lower portions are integrally formed to be shaped as a plate. A winged portion may be disposed below the lower inlet port. The capacitive sensor may not extend above the upper inlet port.

In certain implementations, the PCB unit is disposed within an electronics compartment. The electronics connector disposed on a rear barrier of the housing. A first wire lead or harness may provide electrical communication between the sensor and the PCB unit. The first wire lead or wire harness extends through a slot in the housing between the sensor compartment and the electronics compartment. The optical sensor may be disposed within the housing with a second wire lead or wire harness providing electrical communication between the optical sensor and the PCB unit. The second wire lead or wire harness may extend through a sealed grommet in the housing to the electronics compartment.

According to another aspect of the present disclosure, the filter assembly includes the housing defining a suction outlet, and one or more inlet ports configured to receive a suction tube. The housing includes the filter casing defining the suction outlet. The filter is disposed within the housing between the one or more inlet ports and the suction outlet. The shielding cover is coupled to the filter casing and includes conductive material configured to provide an electromagnetic barrier. The shielding cover may include a port panel defining the one or more inlet ports. The port panel may not be shielded. In other words, the conductive material may not cover the port panel. The shielding cover may include the inner casing, the outer casing, and the conductive layer of the conductive material disposed between the outer casing and the inner casing. The conductive layer may be heat staked to the inner casing.

In certain implementations, the contact pad is coupled to the housing and configured to engage one or more terminals of the smoke evacuation unit. The conductive layer of the shielding cover is in electrical communication with the contact pad. The conductive bridge may directly contact each of the contact pad and the conductive layer so as to provide electrical communication therebetween. In one example, the conductive layer includes a conductive flange heat staked to an end of the conductive bridge. The conductive bridge may include at least one bend configured to traverse the edge geometry of the housing. Through the conductive bridge and the contact pad, in certain implementations, the conductive material of the shielding cover is configured to be grounded; i.e., electrically coupled to ground such as protective earth.

In another aspect of the present disclosure, the filter receptacle includes one or more terminals. The filter assembly includes defines the inlet ports configured to receive the suction tube, and includes the rear side defining the suction outlet. The filter is disposed within the housing between the inlet ports and the suction outlet. The filter assembly includes the electronic connector coupled to the rear side and configured to be coupled to the electronic connector of the filter receptacle. The contact pad is coupled to the housing. The contact pad is conductive and configured to engage the one or more terminals of the filter receptacle, for example, with the filter assembly fully disposed within the filter receptacle.

In certain implementations, the housing further includes the cover, and sides extending between the rear side the cover. One of the sides includes the step defining the upper portion that is recessed from the lower portion. The contact pad may be coupled to the upper portion of the side. The upper portion of the side defines the one or more windows, the contact pad is exposed through the windows. The upper portion may also include the ridge configured to be engaged by a complementary ridge of the one or more terminals of the smoke evacuation unit to provide defeatable retention of the filter assembly within the filter receptacle. The ridge may define a portion of the windows, and may further extend between the step and an upper one of sides of the housing. In certain implementations, the housing may include a rib separating the contact pad into an upper portion and a lower portion, and wherein the one or more terminals are two terminals spaced apart from one another and configured to engage a respective one of the upper portion and the lower portion of the contact pad.

In certain implementations, the filter assembly may include the liquid separator coupled to the housing. The surgical smoke is directed about a lower edge of a barrier such that momentum causes liquid entrained with the surgical smoke to descend into the liquid collection compartment. One or more port covers may be selectively disposed over a respective one of the one or more inlet ports. The port covers include conductive material configured to provide an electromagnetic barrier. The housing may define a cavity sized to form a handle for grasping the filter assembly. The handle may be positioned opposite the port panel.

In still another aspect of the present disclosure, the housing includes the filter casing defining the filter media compartment. The cover is coupled to the filter casing to define a smoke ingress compartment. The cover may define the inlet ports. The filter is disposed within the filter media compartment. The housing includes the partition to provide fluid separation between the liquid collection compartment disposed beneath the filter media compartment. The partition may support the filter within the filter media compartment. In certain implementations, the partition may be angled or extend horizontally from the rear side of the filter casing. The liquid collection compartment may extend along the length of the filter assembly between the cover and the rear side. The sorbent may be disposed in liquid collection compartment. The sorbent may include a superabsorbent polymer layer and/or an antimicrobial agent. In certain implementations, a side of the housing that defines the liquid collection compartment may further define a detection window. The detection window is sized to permit visualization of liquid collection within the liquid collection compartment. Additionally or alternatively, a liquid sensor may be disposed within the liquid collection compartment.

Additional aspects of the present disclosure are directed to the system. The system may include any implementations of the filter assembly disclosed herein, as well as the smoke evacuation unit and/or the electrosurgical instrument or unit. The electrosurgical instrument configured to be coupled to an electrosurgical unit by the power cord, wherein a change in alternating current supplied to the electrosurgical instrument through the power cord is cause detectable changes in the electric field.

In certain implementations, the filter assembly includes the housing, the electronic connector coupled to the housing, and the capacitive sensor in electronic communication with the electronic connector. The smoke evacuation unit includes a vacuum source, the filter receptacle, the electronic connector disposed within the filter receptacle, and a controller in electrical communication with the vacuum source and the electronic connector. The controller is configured to receive signals from the sensor indicative of changes in electric field as being detected through one or more inlet ports by the capacitive sensor. The controller is further configured to activate, deactivate, or augment operation of the vacuum source based on the changes in the electric field. In one example, the controller activates the vacuum source based on the changes in the electric field being indicative of activation of a powered surgical device. Conversely, the controller may deactivate the vacuum source based on the changes in the electric field being indicative of deactivation operation of the powered surgical device. The controller may provide for variable control of the vacuum source by increasing or decreasing suction from the vacuum source based on the changes in the electric field being indicative of increasing or decreasing power from a powered surgical device, respectively.

In certain implementations, the controller may utilize the signals from the capacitive sensor in combination with additional signals from the optical sensor. In particular, the additional signals may be indicative of particulate being detected as drawn through the filter assembly. The controller may operate the vacuum source to decrease suction to a non-zero level based on the changes in the electric field being indicative of stopping power from a powered surgical device. The controller may then terminate operation of the vacuum source based on a level of the particulate no longer exceeding a predetermined threshold such that minimal or no residual surgical smoke is in the suction tube or the filter assembly.

In certain implementations, the filter assembly may include the contact pad, and the smoke evacuation unit may include the grounding unit comprising one or more terminals. The shielding cover may be in electrical communication with the contact pad. The controller is in electrical communication with the grounding unit. The controller is configured to receive signals from the electronic connector as being coupled to the electronic connector of the filter assembly, and determine a presence or absence of an electrical pathway in which the contact pad is in electrical communication with the one or more terminals of the grounding unit. Based on a determined presence of absence of the electrical communication, the controller may operate or adjust operation of the vacuum source. For example, the controller may prevent or limit operation of the vacuum source based on a determined absence of the electrical pathway.

Additional inventive aspects are disclosed in the detailed description and accompanying figures.

### BRIEF DESCRIPTION OF THE DRAWINGS

FIG. 1 depicts a filter assembly configured to be removably coupled with a smoke evacuation unit of a medical waste collection system. A power cord of an electrosurgical instrument is positioned near one of plurality of inlet ports of the filter assembly to which a suction tube of the suction instrument is removably coupled.

FIG. 2 depicts another exemplary arrangement of the system in which a console includes the electrosurgical unit and the smoke evacuation unit. The power cord of the electrosurgical instrument is positioned near one of the inlet ports to which the suction tube of the suction instrument is removably coupled.

FIG. 3 is a front right perspective view of a first implementation of the filter assembly configured to be disposed within a filter receptacle of the smoke evacuation unit.

FIG. 4 is a front left perspective view of the filter assembly.

FIG. 5 is a rear perspective view of the filter assembly.

FIG. 6 is front exploded view of the filter assembly.

FIG. 7 is a rear exploded perspective view of the filter assembly.

FIG. 8 is a partial perspective view of the filter assembly in which an electronics cover is exploded to depict a contact pad disposed within the housing, and a conductive bridge configured to provide electrical communication between a shielding cover and the contact pad.

FIG. 9 is a plan view depicting electrical contact between subcomponents of the filter assembly and the filter receptacle.

FIG. 10 is a sectional elevation view of the filter assembly of FIG. 5 taken along lines 10-10.

FIG. 11 is a partially exploded front perspective view of a second implementation of the filter assembly.

FIG. 12 is a front left exploded view of the filter assembly.

FIG. 13 is a front right exploded view of the filter assembly.

FIG. 14A is a perspective view of a first variant of a capacitive sensor. Relative locations of the inlet ports of the filter assembly are shown in phantom.

FIG. 14B is a perspective view of a second variant of the capacitive sensor. Relative locations of the inlet ports of the filter assembly are shown in phantom.

FIG. 15 is a front exploded perspective view of a third implementation of the filter assembly.

FIG. 16 is a partially exploded rear perspective view of the filter assembly in which the shielding cover is separated from the housing to depict a sensor compartment and smoke ingress compartment.

FIG. 17 is a sectional elevation view of the filter assembly.

FIG. 18 is a partially exploded front perspective view of a fourth implementation of the filter assembly in which the shielding cover is separated from the housing.

FIG. 19 is a rear perspective view of the filter assembly.

FIG. 20 is a sectional elevation view of the filter assembly of FIG. 19 taken along section lines 20-20.

FIG. 21 is a schematic representation of electronic components of the smoke evacuation unit and the filter assembly. Optional components and connections of certain variants are indicated with dashed lines.

FIG. 22A is a front perspective view of another implementation of the filter assembly in which terminals disposed on the housing are in electrical communication with the shielding cover.

FIG. 22B is a front perspective view of an implementation of the filter assembly in which terminals associated with the electric connector are in electrical communication with the shielding cover.

FIGS. 23A-23C are schematic representations of a first variant of electrical pathways between the filter assembly and the controller of the smoke evacuation unit.

FIGS. 24A-24C are schematic representations of a second variant of electrical pathways between the filter assembly and the controller of the smoke evacuation unit.

FIGS. 25A and 25B are schematic representations of a third variant of electrical pathways between the filter assembly and the controller of the smoke evacuation unit.

FIG. 26 is a schematic representation of a fourth variant of electrical pathways between the filter assembly and the controller of the smoke evacuation unit.

FIG. 27 is a schematic representation of a fifth variant of electrical pathways between the filter assembly and the controller of the smoke evacuation unit.

### DETAILED DESCRIPTION

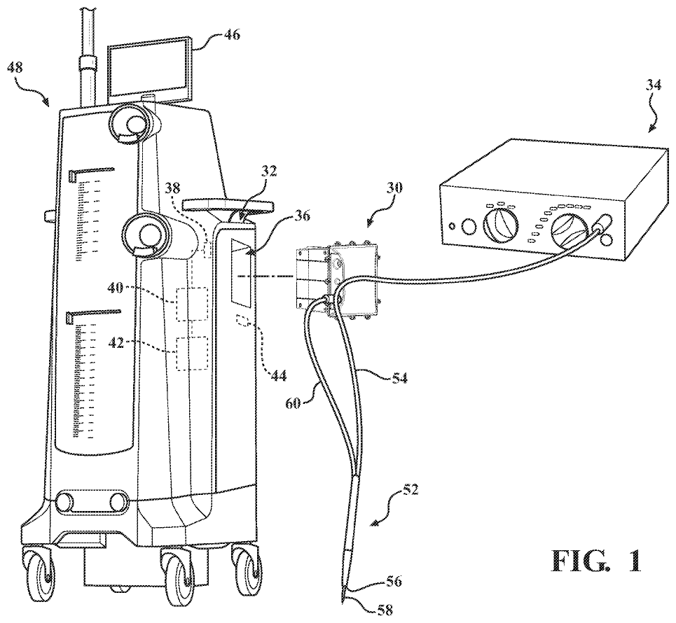

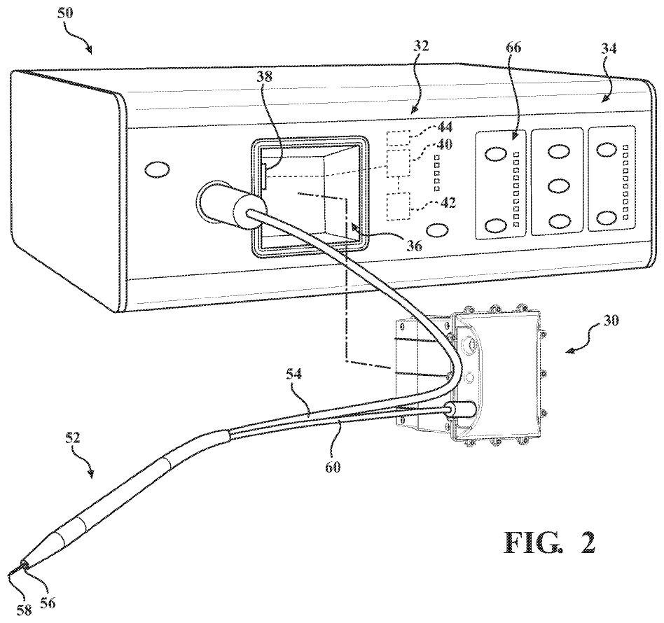

FIGS. 1 and 2 illustrate exemplary arrangements of a filter assembly 30 for use with a smoke evacuation unit 32, and an electrosurgical unit 34 or other powered surgical unit. The smoke evacuation unit 32 includes a filter receptacle 36 within which the filter assembly 30 is configured to be removably inserted or disposed. The smoke evacuation unit 32 also includes an electronic connector 38, a controller 40, a vacuum source 42, and, optionally, a reference antenna 44. The controller 40 is in electronic communication with the electronic connector 38 and the vacuum source 42. In manners to be further described, the controller 40 is configured to control the vacuum source 42 based on the data being received from the filter assembly 30 through the electronic connector 38. The data and controllable features of the smoke evacuation unit 32 may be displayed on a display 46, which may include a user interface.

FIG. 1 depicts the smoke evacuation unit 32 integrated on a medical waste collection system 48, for example, a wheeled cart supporting one or more waste canisters for separately collecting liquid medical waste under the influence of suction. The display 46 of the medical waste collection system 48 may be a graphical user interface (GUI) configured to control operations of the smoke evacuation unit 32. Additional subsystems and functionality of the medical waste collection system 48 are disclosed in the aformentioned United States Patent Publication No. 2007/0135779, as well as commonly owned International Patent Publication No. WO2017/112684, published Jun. 29, 2017, and International Publication No. WO2020/209898, published Oct. 15, 2020, the entire contents of each being hereby incorporated by reference. The electrosurgical unit 34 is provided as a console separate from the medical waste collection system 48. FIG. 2 depicts an alternative arrangement in which a console 50 includes the smoke evacuation unit 32 and the electrosurgical unit 34. One suitable console providing the dual functionality is the SafeAir Compact Evacuator sold by Stryker Corporation.

The electrosurgical unit 34 is configured to operate an electrosurgical instrument 52 to, among other things, cauterize, resect, or augment tissue with electrical energy. The electrosurgical instrument 52 is coupled to the electrosurgical unit 34 with a power cord 54. Further, in exemplary implementations, the electrosurgical instrument 52 defines a suction aperture 56 providing for evacuation of the surgical smoke. The suction aperture 56 may be adjacent to a tip 58 configured to deliver the electrical energy to the tissue. In other words, the suction instrument and the electrosurgical instrument may be structurally integrated, for example, on a pencil-shaped device, such as the SafeAir Smoke Evacuation Pencil sold by Stryker Corporation and disclosed in commonly owned International Patent Publication No. WO2013/000465, published Jan. 3, 2013, the entire contents of which are hereby incorporated by reference. Alternatively, the suction instrument may be separate from the powered surgical device.

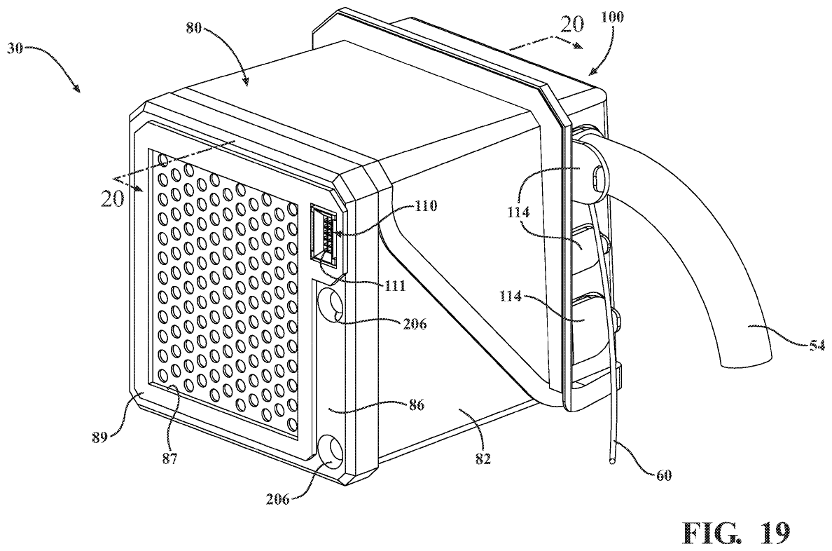

A suction tube 60 couples the electrosurgical instrument 52 to the filter assembly 30, and the power cord 54 couples the electrosurgical instrument 52 to the electrosurgical unit 34. The power cord 54 may be routed to be positioned near or adjacent the filter assembly 30. An exemplary arrangement is representatively shown in FIGS. 1 and 2 in which the power cord 54 is positioned proximate to where the suction tube 60 is coupled to the filter assembly 30 (see also FIG. 19). In one non-limiting example, the power cord 54 may be disposed within the suction tube 60, or each within an overlying sheath, and exit the suction tube 60 immediately prior to where the suction tube 60 is coupled to the filter assembly 30, thereby facilitating the power cord 54 being appropriately positioned near the filter assembly 30. Such an arrangement is disclosed in the aformentioned International Patent Publication No. WO2013/000465. In another example, a clip or other suitable retention feature may be provided to support the power cord 54 in a position adjacent or proximate to the filter assembly 30.

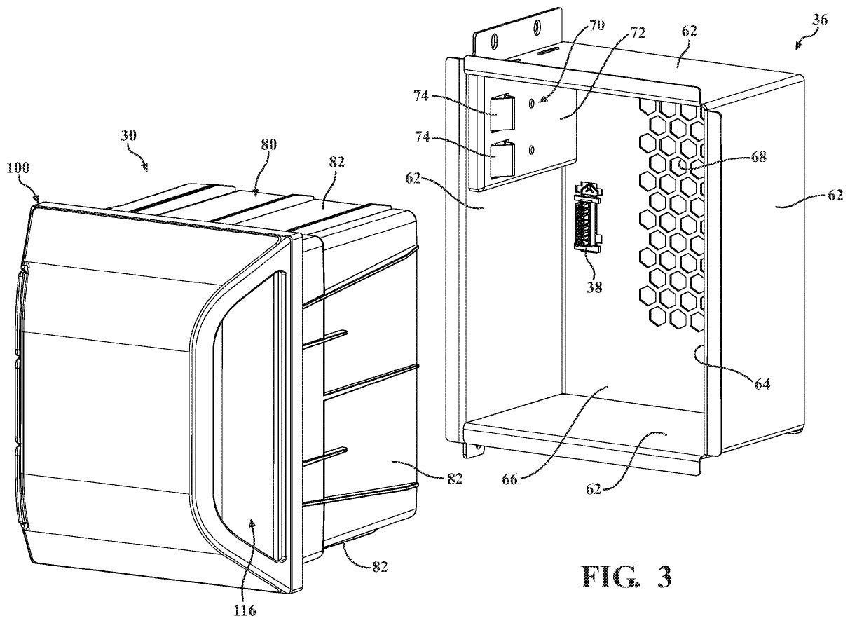

Referring to FIG. 3, the receptacle 36 may be formed by a plurality of side barriers 62 defining a filter opening 64, and a rear barrier 66. The rear barrier 66 defines an outlet opening 68 through which suction is drawn on the filter assembly 30 disposed therein. The outlet opening 68 is in fluid communication with the vacuum source 42 through suitable ducting, conduits, or the like. The outlet opening 68 is depicted as a screen or grate, but alternative configurations are contemplated. The electronic connector 38 may be disposed on or coupled to the rear barrier 66 of the receptacle 36. The electronic connector 38 may be, for example, a plug including a plurality of pins through which power, data, and/or other signals are configured to be transmitted to and from the filter assembly 30. For reasons to be described, each of the barriers 62, 66 may be formed from conductive material to provide an electromagnetic (EM) barrier configured to limit, dampen, reduce, or prevent changes in electric field therethrough. For example, the barriers 62, 66 of the receptacle 36 may be formed from sheet metal. Therefore, other than through the filter opening 64, EM noise or signals generally may not pass through the receptacle 36, also referred to herein as a shielded receptacle. The receptacle 36 may be mounted within the medical waste collection system 48 or the console 50 through suitable means, for example, fasteners engaging flanges extending from one or more of the barriers 62, 66.

In certain implementations, the receptacle 36 includes a grounding unit 70. FIG. 3 depicts the grounding unit 70 including a casing 72 coupled to one of the side barriers 62 and generally positioned in an upper side corner of the receptacle 36. Other locations for the casing 72 within the receptacle 36 are possible, and there may be more than one grounding unit. The casing 72 may be secured to the barrier(s) 62 with fasteners or other suitable joining means. The casing 72 defines one or more openings through which one or more terminals 74 are exposed. The illustrated implementation includes an upper terminal and a lower terminal generally aligned vertically. Other suitable arrangements are contemplated, including but not limited to horizontal alignment, horizontal and vertical staggering, and the like. The terminals 74 are formed from conductive material and arranged in electronic communication with the controller 40 of the smoke evacuation unit 32. The terminals 74 may be spring-biased outwardly and configured to resiliently deflect inwardly. In manners to be further described, the terminals 74 are configured to engage at least one contact pad 76 of the filter assembly 30 to electrically ground certain conductive components of the filter assembly 30. Further, doing so may enable the controller 40 to determine that the filter assembly 30 includes the conductive subcomponents, and therefore is a genuine filter assembly.

Referring to FIGS. 3-5, the filter assembly 30 includes a housing 80. The housing 80 may include a plurality of sides 82, 84 and a rear side 86 arranged in a shape generally complementary to a shape of the filter opening 64 of the receptacle 36. The illustrated implementation shows the sides 82, 84, 86 providing a box-shaped form factor to the housing 80. Other complementary geometries are contemplated, including circular, elliptical, rectangular, and higher-order polygonal shapes. One or more of the sides 82, 84 may include ribs or other protrusions to facilitate alignment with insertion and/or provide a defeatable friction fit between the filter assembly 30 and the barriers 62 of the receptacle 36. Among other advantages, the arrangement prevents inadvertent jostling or decoupling of the filter assembly 30 as, for example, the medical waste collection system 48 is wheeled about the medical facility.

The rear side 86 of the housing 80 may be complementarily shaped to the rear barrier 66 of the receptacle 36. The illustrated implementation shows the rear side 86 and the rear barrier 66 being substantially flat or planar. The rear side 86 of the filter assembly 30 defines a suction outlet 87 sized and shaped complementary to the outlet opening 68 of the receptacle 36, and a seal 89 may be disposed about the suction outlet 87 to facilitate a sealed pathway between the suction outlet 87 and the outlet opening 68. The seal 89 may be a rope or cord seal protruding proximal to the rear side 86 so as to directly contact the rear barrier 66 with the filter assembly 30 fully inserted into the receptacle 36.

One of the sides 84 may include a step 88 extending inwardly to define a cutout or recess, which may be defines an upper portion 90 of the side 84 and a lower portion 92 of the side 84. More particularly, the step 88 defines the recess extending distally or forward from the rear side 86 to a position distal to the contact pad 76. The size and shape of the recess may be complementary to the size and shape of the casing 72 of the grounding unit 70. As a result, the shape of the rear side 86 may include zero lines of symmetry, and the complementary geometries effectively require the user to insert the filter assembly 30 into the filter opening 64 of the receptacle 36 in a single orientation.

The upper portion 90 of the side 84 defines one or more windows 94 through which the contact pad 76 is exposed. The contact pad 76 is disposed within the housing 80, and more particularly secured to an inner surface of the housing 80 for at least a portion of the contact pad 76 to be exposed through the windows 94. For example, the contact pad 76 may be heat staked to the inner surface. The contact pad 76 is formed from conductive material, for example, a plate of metal. In certain implementations, the upper portion 90 of the housing 80 may include a rib 95 separating the windows 94. The rib 95 may have a thickness approximate to a gap between the terminals 74 of the grounding unit 70. The windows 94 are sized and positioned such that, with the filter assembly 30 fully inserted into the receptacle 36, the terminals 74 of the grounding unit 70 directly contact the contact pad 76. In the exemplary implementation, an upper one of the terminals 74 engages an upper portion of the contact pad 76 through an upper one of the windows 94, and a lower of the terminals 74 engages a lower portion of the contact pad 76 through a lower one of the windows 94. Alternatively, the contact pad 76 may be two, discrete contact pads with a first contact pad engageable through the upper one of the windows, and a second contact pad engageable through a lower one of the windows. The direct contact between the conductive components establishes an electrical pathway between the contact pad 76 to the terminals 74, which will be discussed in greater detail herein.

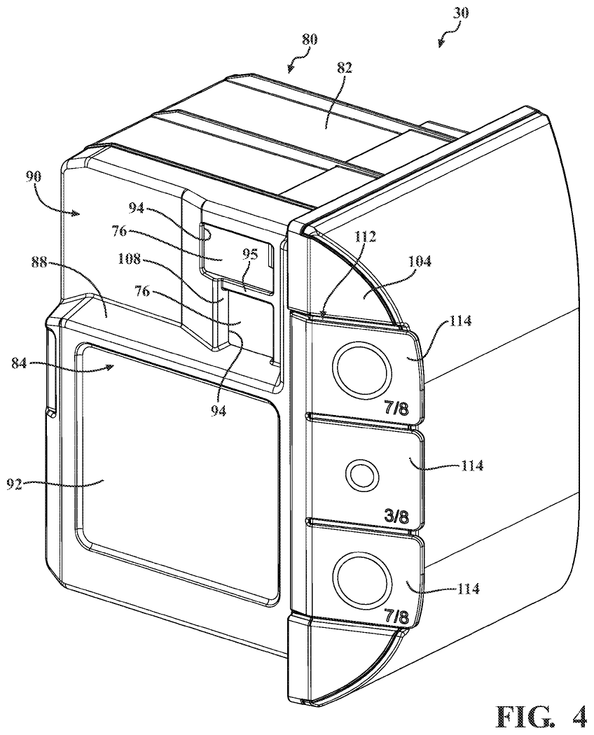

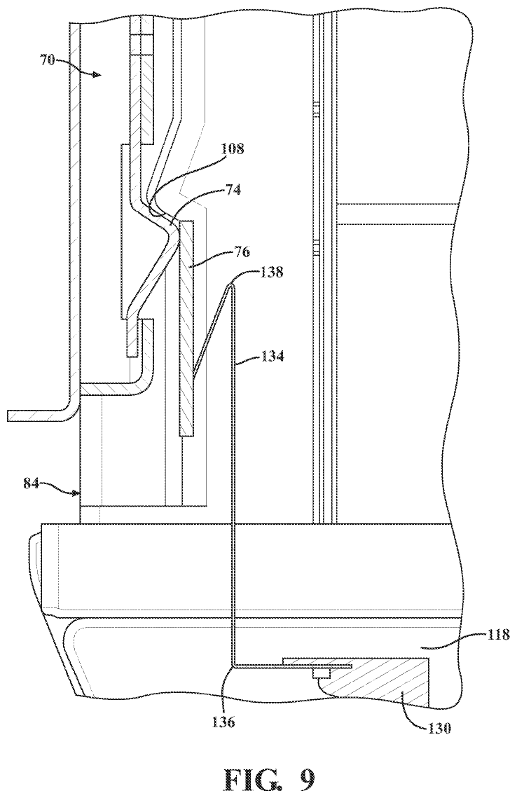

The housing 80 may include a ridge 108 configured to be engaged by the terminals 74 of the receptacle 36. As best shown in FIG. 4, the ridge 108 is formed on the upper portion 90 of the side 84 and defines a portion of at least one of the windows 94. The ridge 108 is oriented vertically and extends between the step 88 of the side 84 and the upper side 82 of the housing 80. The rib 95 may extend distally or forward from the ridge 108. Engagement between the terminals 74 and the ridge 108 provide defeatable retention of the filter assembly 30 within the receptacle 36. In particular, the slope of the ridge 108 is configured to be engaged by the V-shaped profile of the terminals 74 (see FIG. 9). With a pulling input provided to a handle 116 of the filter assembly 30, the terminals 74 may resiliently deflect slightly within the casing 72 of the grounding unit 70, after which the filter assembly 30 may be removed from the receptacle 36.

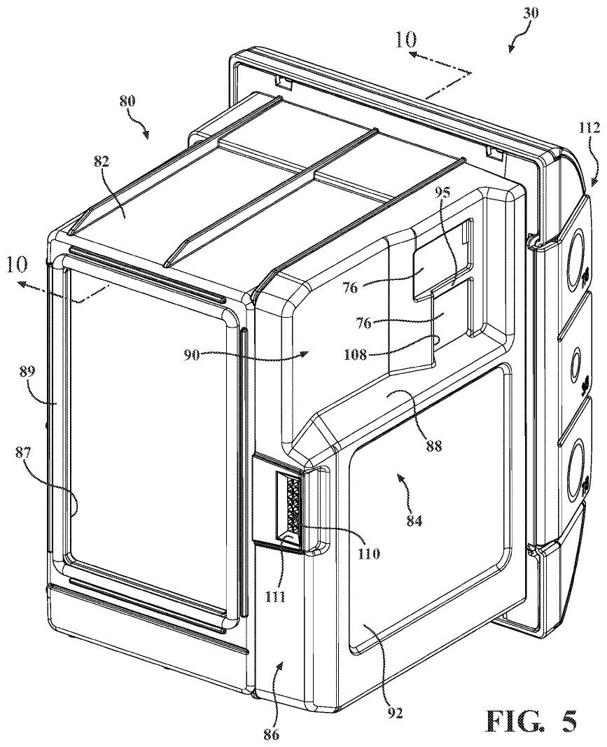

An electronic connector 110 may be coupled to and disposed on the rear side 86 of the filter assembly 30. The electronic connector 110 is configured to provide electronic communication with the electronic connector 38 of the smoke evacuation unit 32 with the filter assembly 30 disposed within the receptacle 36. The electronic connector 38 may be a female coupler (e.g., a socket), as shown, or may be a male coupler (e.g., a plug). FIG. 5 shows the electronic connector 110 including pin receptacles configured to receive the pins of the electronic connector 38 of the smoke evacuation unit 32 to facilitate the transmission of power, data, and/or other signals therebetween. To account for any slight tolerancing variances, the electronic connector 110 may include at least one ramped surface 111 configured to facilitate a connection with the electronic connector 38 of the smoke evacuation unit 32 as the filter assembly 30 is being directed to within the receptacle 36. Additionally or alternatively, alignment features 206 may be used to facilitate the connection (see FIG. 19).

The housing 80 may include a filter casing 98, a shielding cover 100 coupled to the filter casing 98 to collectively form the enclosure, including several compartments therein to be described. A sensor 102 is disposed within the enclosure of the housing 80. The sensor 102 is configured to facilitate control of the vacuum source 42 of the smoke evacuation unit 32 based on energizing, deenergizing, and adjusting energy provided to the electrosurgical instrument 52 (or other powered surgical instrument or device). In particular, the sensor 102 is configured to detect alternating current (AC) flowing through the power cord 54 of the electrosurgical instrument 52 by detecting changes in the electric field external to the power cord 54. The shielding cover 100 is configured to reduce, dampen, limit, or prevent potential EM noise from being detectable by the sensor 102 within the housing 80, thereby preventing inadvertent activation of the vacuum source 42. In other words, the sensor 102 may be tuned such that the detected changes in the electric field through the inlet ports 104 are of sufficient magnitude to be attributable as the power cord 54, as opposed to other powered surgical device less proximate to the filter assembly 30.

The shielding cover 100 may be removably coupled or secured to the filter casing 98, for example, with fasteners, clips, laser or ultrasonic welding, or other suitable joining means. A gasket may be disposed at an interface between the shielding cover 100 and the filter casing 98. The shielding cover 100 is sized slightly larger than the housing 80 so as to form a lip or flange configured to abut a front edge of the barriers 62 of the receptacle 36 with the filter assembly 30 fully inserted therein. Alternative implementations of the filter assembly 30 are contemplated in which EM-based control of the vacuum source 42 is not provided. In those implementations, the shielding cover 100 is optional, instead forming a cover or a front side of the housing 80.

The shielding cover 100 includes a port panel 104 defining one or more inlet ports 106. Each of the inlet ports 106 are configured to be removably coupled with a suction tube. The illustrated implementations include three suction ports—two configured to receive a ⅞″ suction tube and one configured to receive a ⅜″ suction tube, as indicated by numerical indicia on respective port covers 114, which may also be referred to as flaps, caps, tabs, or the like. The indicia may also include rings sized to the corresponding suction port, color coding, or the like. It is understood that more or less inlet ports may be provided, and the inlet ports may be of any suitable dimension(s). A port cover assembly 112 may be coupled to the port panel 104 and include a spine to which the port covers 114 are flexibly or pivotably coupled. As such, the port covers 114 are movably disposed over a respective one of the inlet ports 106. For example, the port covers 114 may be formed from a flexible material such as a rubber, or a semi-rigid or rigid material that is pivotably coupled to the port panel 104 of the housing 80. The port covers 114 preserve the sealed pathway through the filter assembly 30 by preventing ingress of ambient air through the inlet port(s) 106 not in use. Moreover, the port covers 114 prevent debris or other particles are entering the filter assembly 30. Opposite the port panel 104, the shielding cover 100 may also be formed or shaped to define a cavity sized to provide the handle 116 for a user to manipulate the filter assembly 30, such as installing into or removing from the receptacle 36, or otherwise carrying it about the medical facility.

In optional implementations, the port covers 114 may be at least partially formed from conductive material configured to reduce, dampen, limit, or prevent changes in electric field therethrough. In other words, the port covers 114 may be an EM barrier. Non-limiting example includes a metal-filled rubber, such as a carbon-filled silicone elastomer, a metal-encapsulated member (e.g., via overmolding), metal foil or mesh, conductive plastic, conductive coating such as paint or plating, or combinations thereof. Therefore, the port covers 114—except for those receiving a suction tube (i.e., in an open position)—and particularly in combination with the shielding cover 100, limits or prevents EM energy from passing from the external environment into the housing 80 to be detected by the sensor 102.

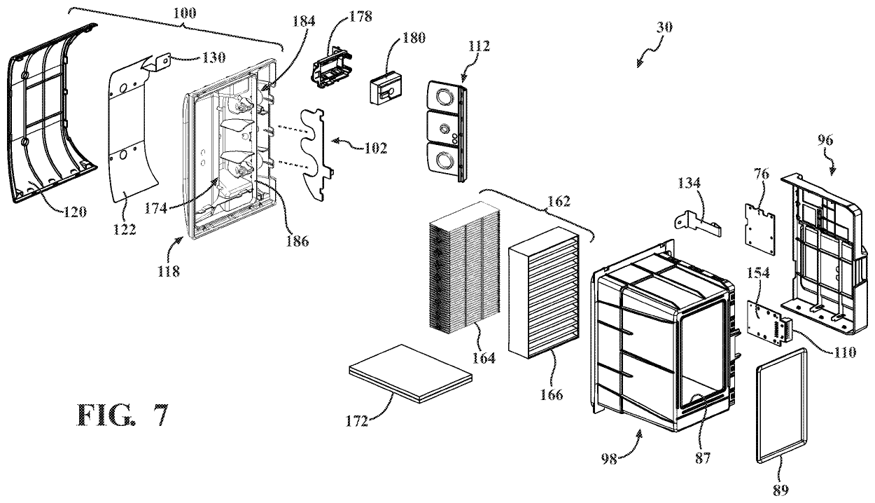

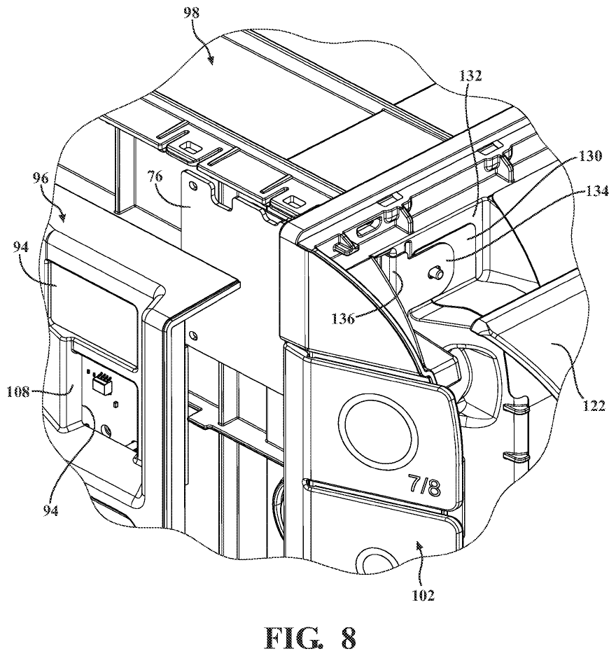

With continued reference to FIGS. 7 and 8, the shielding cover 100 may include an inner casing 118, an outer casing 120, and a shielding layer 122 disposed between the inner casing 118 and the outer casing 120. The shielding layer 122 is at least partially formed from conductive material configured to reduce, dampen, limit, or prevent changes in electric field therethrough. In other words, at least a portion of the shielding cover 100 provides an EM barrier by interfering with, reducing, and/or blocking the transmission of electromagnetic energy or waves from passing from the external environment to within the enclosure of the filter assembly 30. Non-limiting examples of the conductive material include metal, metal foil or mesh, conductive plastic, conductive coating such as paint or plating, or combination thereof.

The shielding layer 122 may be secured to the inner casing 118 and/or the outer casing 120. As shown in FIG. 7, for example, the inner casing 118 includes posts 124 and the shielding layer 122 includes apertures 126 respectively configured to facilitate alignment and heat staking of the shielding layer 122 to the inner casing 118. The outer casing 120 may be secured to the inner casing 118 with snaps, welding, fasteners, or other suitable joining process. As generally appreciated from FIGS. 7 and 8, the shielding layer 122 is contoured to a profile of the inner casing 118, and sized to cover a sufficient portion of the inner casing 118, and therefore a front of the filter assembly 30. In one example, the shielding layer 122 is sized to cover nearly an entirety of the inner casing 118 other than the port panel 104.

In one variant, the shielding layer 122 is optional, and the inner casing 118 or the outer casing 120 may be at least partially formed from the conductive material (e.g., embedded or impregnated therein). In another variant, one of the inner casing 118 or the outer casing 120 is optional, and the shielding layer 122 is coupled to, for example, an outer surface of the inner casing 118 or an inner surface of the outer casing 120. In still another variant, the cover is unitary in construction and formed from the conductive material (e.g., formed metal).

As mentioned, the sensor 102 is configured to detect changes in the electric field (e.g., changes in radiofrequency (RF) or other EM energy). In a preferred implementation, the sensor 102 is a capacitive sensor, also considered an antenna, reader, transceiver, e-field sensor, or the like. Other types of sensors configured to wirelessly detect changes in energy characteristics through the inlet ports 106 are within the scope of the present disclosure. The sensor 102 is disposed within the housing 80, for example, coupled to or disposed proximate to the shielding cover 100. The positioning of the sensor 102 on or near the shielding cover 100 may be based on achieving the desired signal to noise ratio to detect the changes in the electric field across a range of expected operating scenarios. The sensor 102 may be coupled to or positioned adjacent to the port panel 104, as best shown from FIG. 7, as to be detect the changes in the electric field through the inlet ports 106. More specifically, the inner casing 118 of the shielding cover 100 may define a sensor compartment 184 within which the sensor 102 is configured to be slidably inserted during assembly of the filter assembly 30. The sensor compartment 184 may include at least one coupling feature configured to support the sensor 102 therein.

As to be further described, the sensor 102 may be shaped to a footprint of most of the port panel 104 other than the inlet ports 106. With the shielding cover 100 providing the EM barrier to most or all of the front of the filter assembly 30 other than the port panel 104 (and with the receptacle 36 being shielded), the changes in the electric field are essentially detectable only through the inlet ports 106. Therefore, the changes in the electric field, as detected by the sensor 102, are attributable to changes in AC from the power cord 54—positioned near the port panel 104—as the electrosurgical instrument 52 is activated, deactivated, and power adjusted during use.

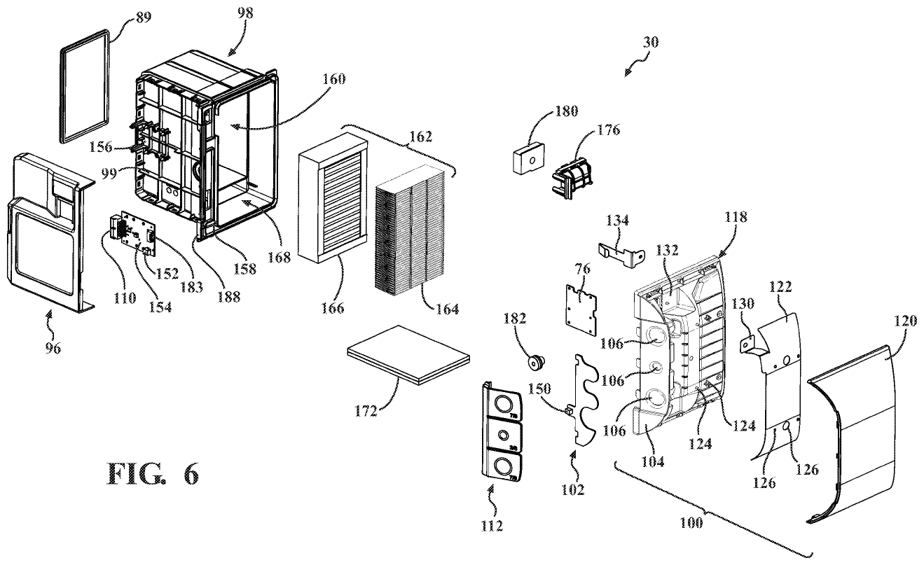

The EM barrier may be further facilitated by the shielding cover 100 being grounded to protective earth, for example, through the contact pad 76 engaging the grounding unit 70 of the receptacle 36. Therefore, the shielding layer 122 of the shielding cover 100 is in electrical communication with the contact pad 76. Referring now to FIGS. 6-9, the shielding layer 122 may include a conductive flange 130. The conductive flange 130 may be positioned within a casing recess 132 of the inner casing 118. The conductive flange 130 may be integrally formed with the shielding layer 122, as shown, or a discrete conductive component coupled thereto. An end of a conductive bridge 134 directly contacts the conductive flange 130. As best shown in FIGS. 8 and 9, the conductive flange 130 and the conductive bridge 134 are heat staked to one another, and to the inner casing 118 within the casing recess 132. The conductive bridge 134 includes at least one bend 136 to traverse an edge geometry of the inner casing 118 such that the conductive bridge 134 further extends along the side 84 of the housing 80. The opposite end of the conductive bridge 134 may also include a bend 138 shaped to cause resilient contact with an inner surface of the contact pad 76. The spring-like effect maintains direct contact between the conductive bridge 134 and an inner surface of the contact pad 76. Alternatively, the conductive bridge 134 may be secured to the contact pad 76 in a manner that provides the electrical communication, for example, through soldering. With the filter assembly 30 fully inserted within receptacle 36, the electrical pathway is established from the shielding layer 122, through the conductive bridge 134, the contact pad 76, the terminals 74, and to protective earth. In addition to facilitating the EM barrier, the electrical response of the filter assembly 30 may be detected to ensure that the filter assembly 30 includes the shielding cover 100, manners of doing so being disclosed in more detail below. In alternative implementations, the grounding to protective earth may be through a grounding pin of the electronic connector 110, a grounding element 195, a capacitor of the filter assembly 30, or other suitable means.

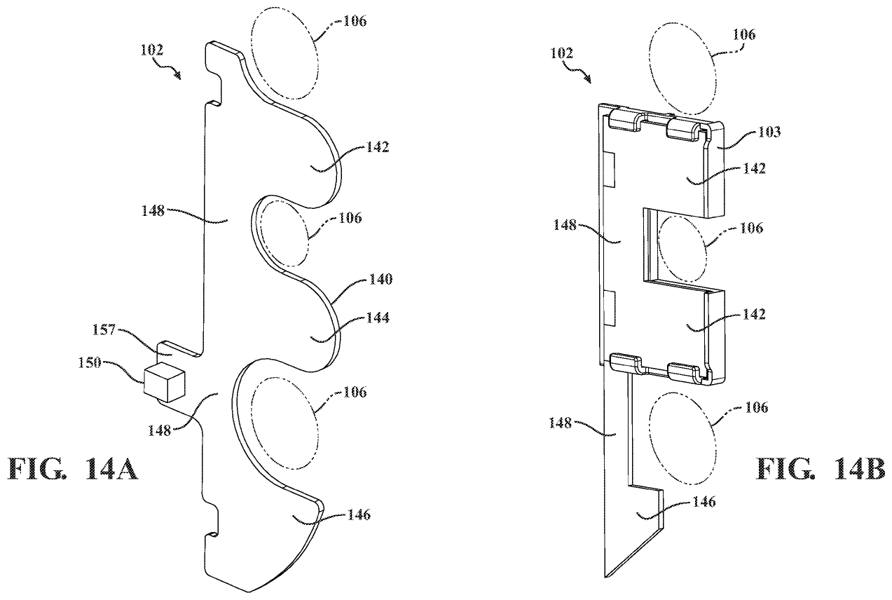

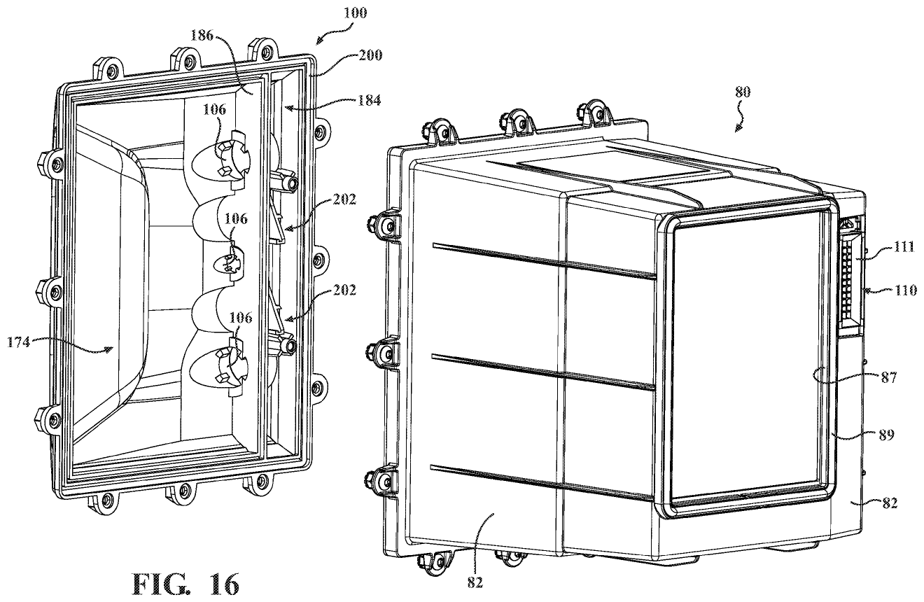

In certain implementations, it may be desirable for the sensor 102 to be maximally sized to increase sensor resolution. As generally appreciated from FIG. 6, the sensor 102 may be sized and shaped to most or nearly an entirety of the port panel 104 (other than the inlet ports 106). The sensor 102 may be disposed on an inner side of the port panel 104 and within a sensor compartment 184 defined by the inner casing 118 to be described (see, e.g., FIG. 16). A first variant of the sensor 102 is shown in FIG. 14A and includes a curvilinear edge 140 contoured to the inlet ports 106. The design provides for portions 142, 144, 146 of the sensor 102 extending near a front edge of the inner casing 118 with the inlet ports 106 remaining exposed. For example, the sensor 102 may include the upper portion 142 disposed above one of the inlet ports 106, and a lower portion 144 disposed below the inlet port 106. In implementation in which there are more than one inlet port, the lower portion 144 may be a middle portion disposed above another one of the inlet ports 106, and a lower or winged portion 146 may be disposed below the other one of the inlet ports 106. Lateral portions 148 may extend between the upper, lower, and winged portions 142, 144, 146. The portions 142, 144, 146, 148 may be integrally formed such that the sensor 102 is plate-like in construction. Alternatively, two, three, or four or more sensors may be provided and suitably positioned within the housing 80. In certain implementations, the sensor 102 may be spaced apart from the conductive material of the shielding cover 100 and the contact pad 76 by a minimum distance so as to limit destruction of the electric field as being sensed by the sensor 102. In other words, the shielding cover 100 provides an EM barrier, as mentioned, and insufficient spacing of the sensor 102 from the shielding cover 100 may result in suboptimal resolution of the sensor 102. The minimum distance may be, for example, at least one-half centimeter or 1, 2 or 3 or more centimeters. For example, as appreciated from FIG. 14A, the sensor 102 does not extend above the upper inlet port 106 so as to maintain electrical separation from the shielding cover 100 and the contact pad 76.

The sensor 102 is in electronic communication with the electronic connector 110 of the filter assembly 30. A wire harness (not shown) may include a first plug coupled to a socket 150 of the sensor 102, and a second plug coupled to a socket 152 of a printed circuit board (PCB) unit 154 to which the electronic connector 110 is coupled (see FIG. 6). The PCB unit 154 may be secured to webbing 156 integrated with an outer surface of the filter casing 98, and a coupling flange 157 of the sensor 102 may be extend through a slot 158 of the filter casing 98 for the wire harness to be routed from the sensor compartment 184 to the PCB unit 154.

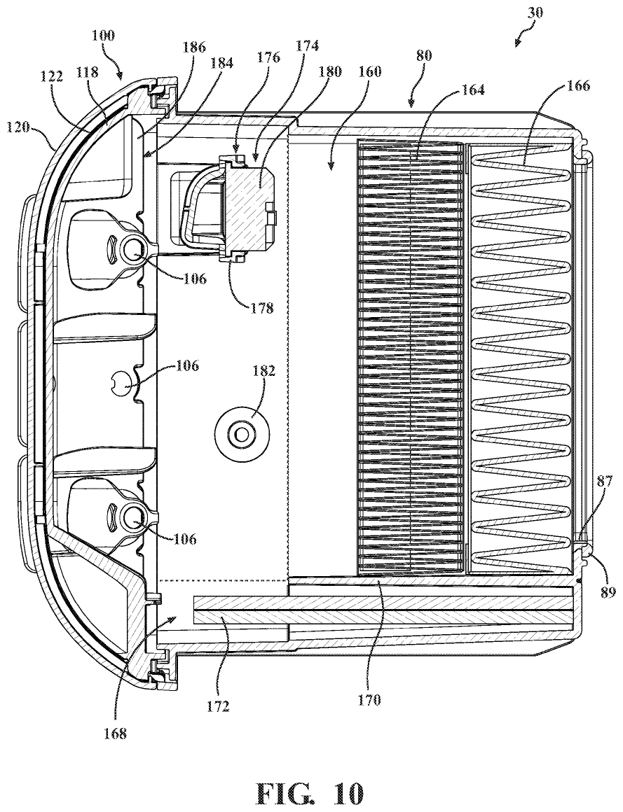

Returning to FIGS. 6 and 7 and with further reference to the sectional view of FIG. 10, the filter casing 98 of the filter assembly 30 defines a filter media compartment 160, and a filter 162 may be disposed within the filter media compartment 160. The filter media compartment 160 may be square or rectangular in section, and the filter 162 complementarily shaped to provide a fluid-tight seal at an interface between outer surfaces of the filter 162 and inner surfaces of the sides of the filter casing 98. The filter 162 may be partially compressed with resiliency of the materials providing the fluid-tight seal, and/or an adhesive or gasket may be disposed at the interface.

The filter 162 may be formed from a plurality of filter layers, also referred to as a filter stack. The filter layers are generally formed from fibrous or porous materials to remove particulates such as dust, pollen, mold, and bacteria from the surgical smoke. One or more of the filter layers may include differing materials or of differing construction so as to filter the smoke passing therethrough in differing manners. For example, a first filter layer (not shown) may be a prefilter of a thick tilter foam configured to capture larger particles. A second filter layer 164 may be an ULPA filter. A third filter layer 166 may be a charcoal filter including a sorbent or catalyst to remove odors from gaseous pollutants such as volatile organic compounds or ozone.

The filter casing 98 of the housing 80 may further define a liquid collection compartment 168 disposed beneath the filter media compartment 160. The liquid collection compartment 168 may also be referred to as a sump, basin, fluid trap, or the like. FIG. 10 shows a partition 170 separating the liquid collection compartment 168 from the filter media compartment 160. The partition 170 may extend from the rear side 86 of the housing 80. The illustrated implementation shows the partition 170 extending horizontally therefrom, but alternatively the partition 170 may be tapered and/or angled. The partition may extend at least 40, 60, 80% or more of a length of the filter assembly defined between the rear side 86 and the cover 100. Therefore, in certain implementations, the position and orientation of the partition 170 provides for the liquid collection compartment 168 extending nearly an entirety of the length of the housing 80, thereby maximizing its liquid capacity. The sectional elevation view of FIG. 10 shows the liquid collection compartment 168 effectively extending from an inner side of the shielding cover 100 to the rear side 86 of the housing 80. In certain implementations, the liquid collection compartment 168 may have a liquid capacity of 100, 150, 200 or more milliliters (mL) of liquid. Furthermore, the filter casing 98 may be formed as a monolithic structure through a suitable polymer molding operation such that a lower portion of the filter casing 98 is devoid of any seam, seal, ultrasonic bond, or parting line, thereby minimizing or preventing leakage or egress of liquid. In certain implementations, a sorbent 172, such as one or more pieces of foam media or super-absorbing fiber or polymer, may be disposed within the liquid collection compartment 168 to limit or eliminate sloshing of liquid therein. The sorbent 172 may be one or more flat pads sized to the dimensions of the liquid collection compartment 168. FIG. 10 shows a portion of the sorbent 172 positioned distal to a distal end of the partition 170. The sorbent may include an antimicrobial agent to limit or prevent odors, and/or a reagent configured to change color when saturated with liquid. In certain implementations, a liquid separator (not shown)—for example, a barrier or other structure to separate liquid entrained within the surgical smoke—to descent into the liquid collection compartment 168.

In certain implementations, one of the sides 82 of the housing 80 that define the liquid collection compartment 168 may define a detection window (not shown) configured to provide a visual gauge of the liquid level within the liquid collection compartment 168. A float may be disposed within the liquid collection compartment 168 with the float visible through the window. Additionally or alternatively, a fluid level sensor may be disposed within the liquid collection compartment 168 and configured to transmit a fluid level signal to and through the electronic connector 110. The float may be the fluid level sensor, or alternatively the fluid level sensor may be a laser distance sensor, an optical sensor, an ultrasound sensor, or the like. In one variant, the optical sensor may be operably coupled to the receptacle 36 with a field of view including the detection window. The optical sensor is configured to monitor the fluid level through the detection window and transmit corresponding signals to the controller 40.

The controller 40 of the smoke evacuation unit 32 may determine the fluid level, and estimate the remaining operational life based thereupon. The estimate may be further based on static numerical correlations, and/or actual rate of use of the filter assembly 30 as stored in memory. The remaining operational life, along with other relevant metrics, may be displayed on the display 46, either automatically with activation of the smoke evacuation unit 32 or in response to user input. An audible and/or visual alarm may be activated if the controller 40 determines that the fluid level within the liquid collection compartment 168 exceeds a predetermined threshold, or a dynamic threshold based on remaining volume available in view of the rate of increase of fluid therein. The alarm may be triggered if the rate of increase of fluid within the liquid collection compartment 168 exceeds a predetermined rate, which may be indicative that the user mistakenly coupled a liquid medical waste suction tube to the filter assembly 30.

Forward or distal to the filter media compartment 160 is a smoke ingress compartment 174. The smoke ingress compartment 174 may also be above the liquid collection compartment 168, as denoted by dashed lines in FIG. 10. The smoke ingress compartment 174 may be considered a manifold through which the incoming surgical smoke is routed to the filter media compartment 160 from the smoke ingress compartment 174. In certain implementations, the filter assembly 30 includes a sensor assembly 176 configured to detect a characteristic of the gas passing through the smoke ingress compartment 174. The sensor assembly 176 includes a sensor housing 178 coupled to an inner wall of the filter casing 98, and a sensor 180 supported by the sensor housing 178. The sensor 180 is in electronic communication with the PCB unit 154, for example, through a wire or a wire harness extending through a sealed grommet 182 to engage a socket 183 of the PCB unit 154. The sensor 180 may be an optical sensor is configured to detect particulates associated with the surgical smoke, and transmit a signal to the PCB unit 154. For example, the sensor 180 may an infrared emitting diode (IRED) and a phototransistor arranged such to detect reflected light of particles in the gas. In alternative implementations, the sensor 180 may be a chemical sensor, electrical sensor, capacitive sensor, or any other sensor capable of detecting a characteristic or property of the gas. Therefore, certain implementations of the filter assembly 30 include at least (or exactly) two sensors, i.e., the capacitive sensor and the optical sensor. The multi-sensor arrangement provides for improved control of the vacuum source 42 of the smoke evacuation unit 32 in manners to be further described.

Referring now to FIGS. 7 and 10, the inner casing 118 of the shielding cover 100 defines the smoke ingress compartment 174, and further defines the sensor compartment 184. The shielding cover 100 includes a partition 186 separating the sensor compartment 184 from the smoke ingress compartment 174, and the inlet ports 106 may extend through the partition 186 to bypass the sensor compartment 184 and open into the smoke ingress compartment 174. Further, a lateral flange 188 of the filter casing 98 (see FIGS. 3 and 4) cooperates with the partition 186 to seal the sensor compartment 184 from the smoke ingress compartment 174. The shielding cover 100 may include the coupling features to support the sensor(s) 102 within the sensor compartment 184.

In certain implementations, the housing 80 may include an electronics cover 96 coupled to a side of a filter casing 98 to define an electronics compartment 99. The electronics cover 96 may form the side 84 of the housing 80, including the step 88. The lateral flange 188 may define a front barrier of the electronics compartment 99. The contact pad 76 may be secured to the inner surface of the electronics cover 96 so as to be positioned within the electronics compartment 99. The PCB unit 154 may be supported by the webbing 156 within the electronics compartment 99. The sealed grommet 182—with the wire harness extending therethrough—may provide a seal between the electronics compartment 99 and the filter media compartment 160. The arrangement of the several compartments 160, 168, 174, 184 maximizes the volume of the filter media compartment 160 and thus the filtering capacity of the filter assembly 30 for a given footprint or size.

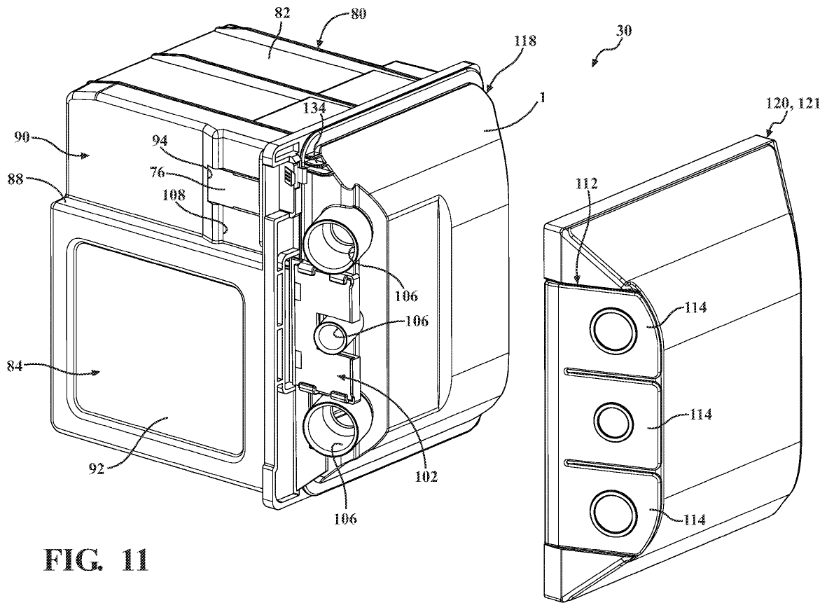

FIGS. 11-13 depict another implementation of the filter assembly 30 with like numerals indicating like components. As with subsequently disclosed implementations of the filter assembly 30, abbreviated discussion of the like components (or those not reintroduced) is in the interest of brevity and should not be construed as limiting. The housing 80 includes the sides 82, 84 and the rear side 86 arranged in a shape complementary to a shape of the filter opening 64 of the receptacle 36. One of the sides 84 includes the step 88 extending inwardly to define a cutout or recess. The size and shape of the recess may be complementary to the size and shape of the casing 72 of the grounding unit 70. The upper portion 90 of the side 84 defines one or more windows 94. The contact pad 76 is exposed through the window(s) 94. The ridge 108 providing the defeatable retention of the filter assembly 30 is oriented vertically and is disposed on each side of the contact pad 76.

The filter assembly 30 includes the shielding cover 100. The shielding cover 100 includes the inner casing 118, the outer casing 120, and an intermediate casing 121. The shielding layer 122 is disposed between the inner casing 118 and the intermediate casing 121. The intermediate casing 121 includes the port panel 104 defining the inlet ports 106. Opposite the port panel 104, the shielding cover 100 may also be formed or shaped to define the cavity sized to provide the handle 116. The cavity is defined by the inner casing 118, and the intermediate casing 121 defines an opening sized to the cavity. The shielding layer 122 of the shielding cover 100 is in electrical communication with the contact pad 76. The shielding layer 122 may include the conductive flange (not identified), and the conductive bridge 134 direct contacts the conductive flange and the inner surface of the contact pad 76.

The filter assembly 30 includes the sensor 102, in particular the capacitive sensor. The sensor 102 is disposed within the housing 80 and coupled to or disposed proximate to the shielding cover 100. More specifically, a cradle 103 may be secured to the inner casing 118, and the cradle 103 supports the sensor 102. The sensor 102 is disposed on an inner side of the port panel 104 and in a void defined by the inner casing 118 (i.e., the sensor compartment 184). With further reference to FIG. 14B, the sensor 102 may include the upper portion 142 disposed above one of the inlet ports 106, the lower portion 144 disposed below the inlet port 106, and winged portion 146 may be disposed below the other one of the inlet ports 106. The winged portion 146 is contoured differently than the implementation previously discussed. The cradle 103 includes tabs configured to secure the sensor 102 to the housing 80. Like the previous implementation, the sensor 102 may not extend above the upper inlet port 106 so as to maintain electrical separation from the shielding cover 100 and the contact pad 76. The sensor 102 is in electronic communication with the electronic connector 110.

The filter casing 98 of the housing 80 defines the filter media compartment 160, and the filter stack 162 may be disposed within the filter media compartment 160. The first filter layer 163 may be a prefilter, the second filter layer 164 may be the ULPA filter, and the third filter layer 166 may be a charcoal filter. The filter casing 98 may further define the liquid collection compartment 168 extending nearly an entirety of the length of the housing 80. A sorbent may be disposed within the liquid collection compartment 168.

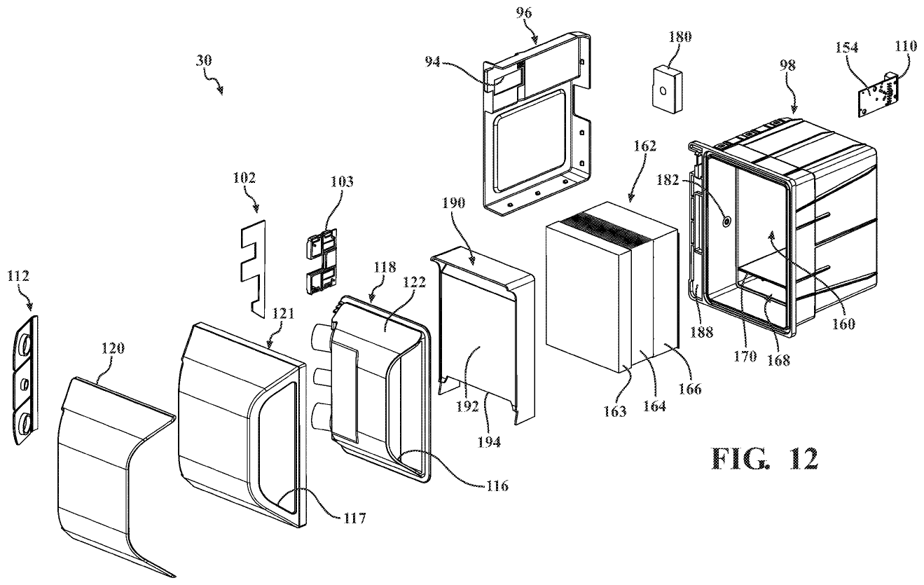

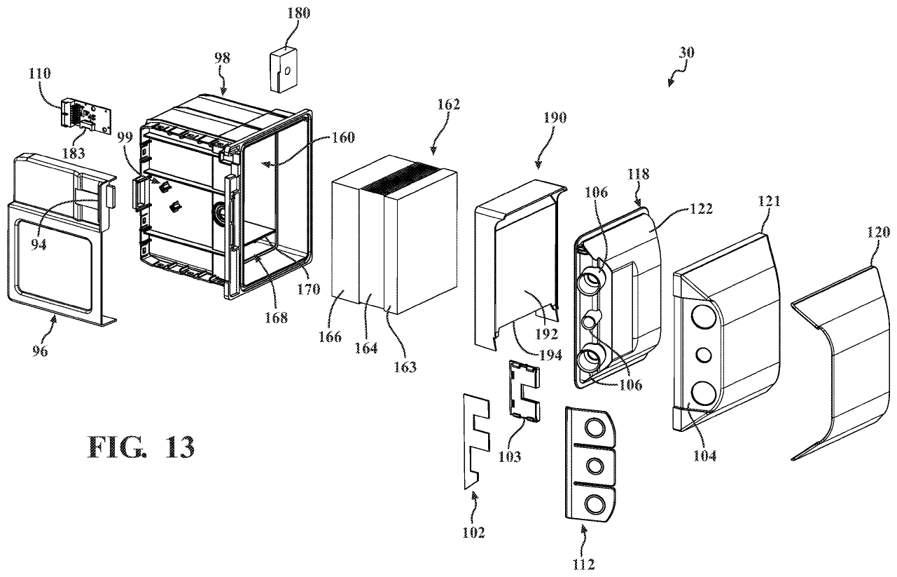

The filter media compartment 160 may be defined on its distal side by a liquid separator 190. The liquid separator 190 is configured to separate liquid entrained within the surgical smoke prior to the fluid encountering the filter 162. With reference to FIGS. 12 and 13, the liquid separator 190 includes a barrier 192 sized and shaped to be supported in the housing 80. The barrier 192 is further sized and shaped to direct gas of the surgical smoke in a tortuous path. In other words, the surgical smoke, including liquid and gas, may encounter the barrier 192, after which the liquid is drawn downwardly along the barrier 192, after which momentum of the fluid about a lower edge 194 of the barrier 192 causes the liquid to be further removed from the gas. Removing the liquid entrained within the surgical smoke advantageously maximizes the filtration efficiency and operational life of the filter assembly 30.

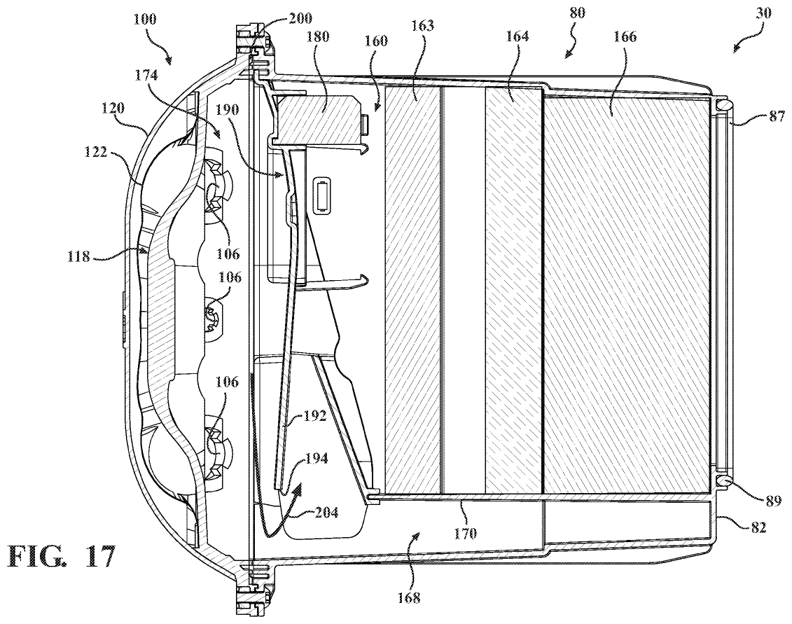

The liquid separator 190 is coupled to the partition 170 to fluidically separate the liquid collection compartment 168 from the filter media compartment 160 (see, e.g., FIG. 17). The liquid separator 190 may also define the smoke ingress compartment 174 opposite the filter media compartment 160. The inlet ports 106 open into the smoke ingress compartment 174. The sensor 180, which is configured to detect a characteristic of the gas passing through the smoke ingress compartment 174, may be coupled to the liquid separator 190. The sensor 180 is in electronic communication with the PCB unit 154, for example, through the wire harness extending through the sealed grommet 182, and into the electronics compartment 99 to engage the socket 183 of the PCB unit 154.

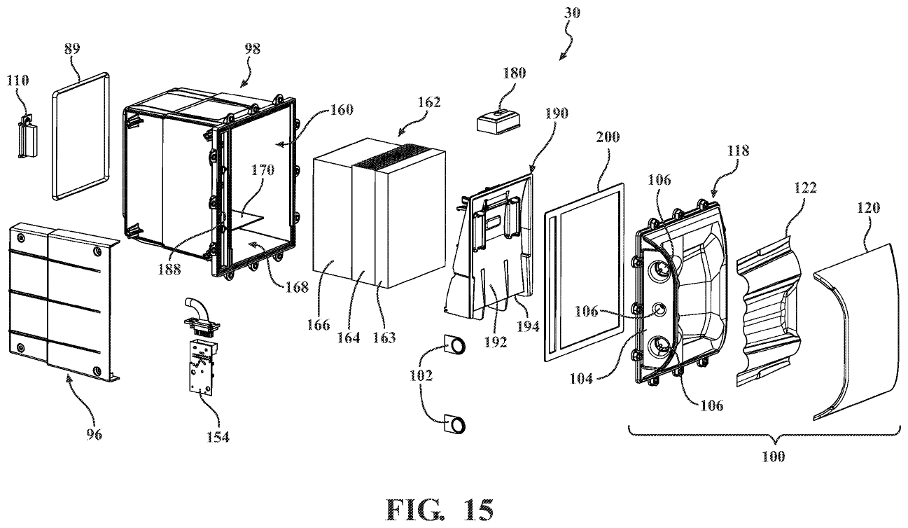

FIGS. 15-17 depict another implementation of the filter assembly 30 with like numerals indicating like components. The housing 80 includes the sides 82, 84 and the rear side 86 arranged in a shape complementary to a shape of the filter opening 64 of the receptacle 36. The housing 80 may not include the contact pad and corresponding structures. The shielding cover 100 includes the inner casing 118, an outer casing 120, and the shielding layer 122. The inner casing 118 includes the port panel 104 defining the inlet ports 106. The inner casing 118 of the shielding cover 100 defines the smoke ingress compartment 174, and a partition 186 fluidly separates the sensor compartment 184 from the smoke ingress compartment 174, and a gasket 200 may be shaped to prevent fluid ingress into the sensor compartment 184.

The filter assembly 30 includes two sensors 102. The sensors 102 are disposed within the housing 80 and coupled to or disposed proximate to the shielding cover 100. More specifically, and with reference to FIG. 16, each of the sensors 102 may be supported in coupling features 202 within the sensor compartment 184. The illustrated implementation depicts two coupling features 202 as rail-shaped protrusions for slidably receiving and supporting one of two sensors 102. Each the two coupling features 202 are disposed between a pair of the three inlet ports 106. The inlet ports 106 bypass the sensor compartment 184. With continued reference to FIG. 16, the inlet ports 106 extend from the port panel 104 and through the partition 186, to open into the smoke ingress compartment 174.

The filter casing 98 defines the filter media compartment 160, and the filter stack 162 may be disposed within the filter media compartment 160. The first filter layer 163 may be the prefilter, the second filter layer 164 may be the ULPA filter, and the third filter layer 166 may be the charcoal filter. The filter casing 98 may further define the liquid collection compartment 168 extending along the length of the housing 80, thereby maximizing its liquid capacity. A sorbent may be disposed within the liquid collection compartment 168.

With reference to FIG. 17, an upper aspect of the liquid separator 190 may be secured in position between the housing 80 and the shielding cover 100. The gasket 200 may be positioned between the housing 80 and the shielding cover 100 to provide a fluid-tight seal therebetween. The liquid separator 190 includes the barrier 192 sized and shaped to direct the gas of the surgical smoke to be directed in the tortuous path 204. The surgical smoke is drawn downwardly along the barrier 192, after which momentum of the fluid about the lower edge 194 of the barrier 192 causes the liquid to be removed from the gas. The liquid separator 190 may engage the partition 170 to fluidically separate the liquid collection compartment 168 from the filter media compartment 160. The sensor 180 is coupled to the liquid separator 190 and configured to detect a characteristic of the gas passing through the smoke ingress compartment 174.

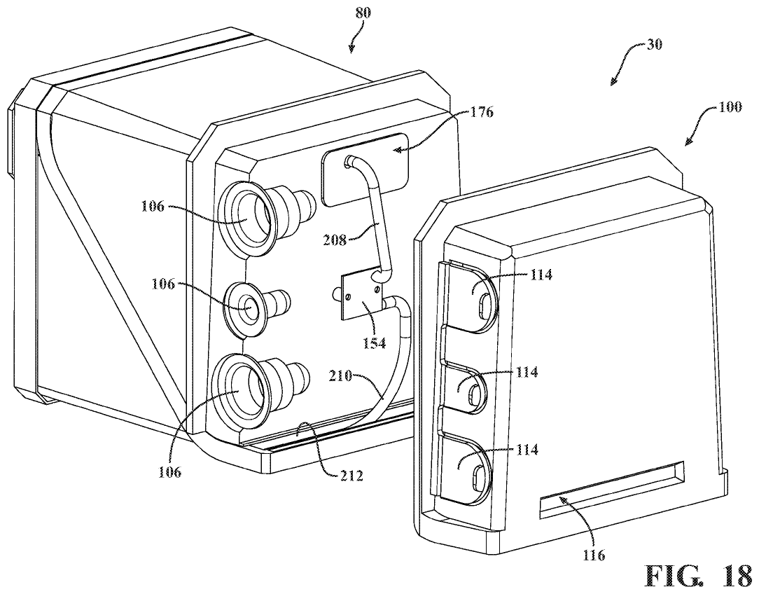

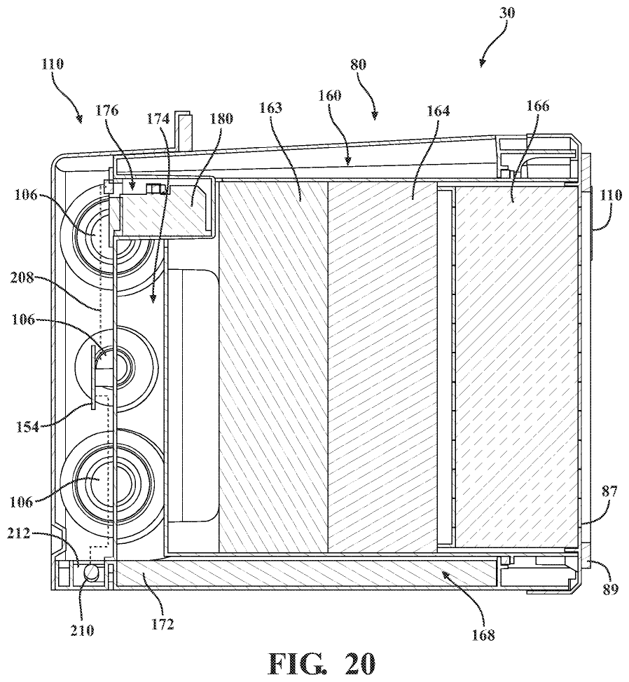

FIGS. 18-20 depict another implementation of the filter assembly 30 with like numerals indicating like components. The implementation includes the housing 80 and the shielding cover 100 having a modified shape to provide a varied overall form factor of the filter assembly 30. FIG. 19 shows the seal 89 surrounding the electronic connector 110 to prevent fluid from compromising its electrical connection with the electronic connector 38 of the receptacle 36. Further, the rear side 86 of the housing 80 may include or define one or more alignment features 206 configured to be engaged by one or more complementary alignment features (not shown) of the receptacle 36. As mentioned, the barriers 62 of the receptacle 36 may be formed from sheet metal, which may be associated with looser mechanical tolerances. In view of the pin-socket engagement of the electronic connectors 38, 110, the alignment features 206 facilitate the requisite alignment. In particular, the alignment features 206 may include a countersunk bore extending inwardly from the rear side 86 of the housing 80. The illustrated implementation shows two countersunk bores vertically aligned and disposed lateral to the suction outlet 87 and below the electronic connector 110. The location is well suited based on the lateral positioning of the electronics compartment 99 to accommodate the countersunk bores extending therein. The complementary alignment features within the receptacle 36 may include one or more posts extending distally from the rear barrier 66. The countersunk bores cooperate with tapered distal ends of the posts to facilitate an alignment, ensuring repeatable coupling of the electronic connectors 38, 110. A retention feature, such as a C-clip, detent, or the like, may be coupled to the alignment features 206 and/or the post to provide a defeatable means of retention of the filter assembly 30 within the receptacle 36. It should be appreciated that the alignment features 206 are optional, or may be an alternative or in addition to the ridge 108 previously described.

The present implementation further differs in the location of the sensor 102, and the inclusion and location of wires 208, 210 (or wire harness) coupling electronic components of the filter assembly 30. FIG. 18 shows the sensor 102 coupled to a front surface of the inner casing 118 of the shielding cover 100. The sensor 102 is approximately centered on the front surface with a detecting surface oriented towards the outer casing 120. The first wire 208 couples the optical sensor 180 to the sensor 102, and the second wire 210 couples the sensor 102 to the PCB unit 154 (not shown). The inner casing 118 may define a cavity or trough 212 sized to receive the second wire 210 and efficiently route the second wire 210 to the electronics compartment. Internal geometries of the housing 80 direct the second wire 210 and the wire harness to be coupled to the electronic connector 110 disposed on the rear side 86 of the housing 80.

Certain inventive aspects of the present disclosure are directed to the implementations of the filter assembly 30. Additional inventive aspects are directed to the smoke evacuation unit 32 (i.e., the system and methods for use with any of the implementations of the filter assembly 30 disclosed herein). Based on activation, deactivation, or adjustment of the power supplied to the electrosurgical instrument 52 or other powered device, the sensor 102 is configured to detect the changes in the electric field through the inlet ports 106, and generate signals therefrom. Likewise, the sensor 180 may be configured to detect particulates associated with the surgical smoke being drawn through the filter assembly 30, and generate signals therefrom. The signals from one or both of the sensors 102, 180 are transmitted to the PCB unit 154. In a first variant, one or more controllers are disposed on the filter assembly 30 and in electronic communication with the PCB unit 154. The PCB unit 154 transmits the signals to the controller(s) for further processing and control to be described. In a second variant, the signals are further transmitted to the controller 40 of the smoke evacuation unit 32 through the electronic connectors 38, 110.

The controller 40 is configured to operate the vacuum source 42 based on determinations from the received signals. In particular, the smoke evacuation unit 32 may be operated in any number of predetermined or configurable modes. In a particulate detection mode, the controller 40 operates the vacuum source 42 d in near-continuous or continuous manner at a lower level. Based on the signals received from the sensor 180 that are indicative of particulates exceeding a predetermined threshold, the controller 40 operates the vacuum source 42 to increase the level of suction. Conversely, once the controller 40 receives signals from the sensor 180 indicative of particulates are below the predetermined threshold, the controller 40 operates the vacuum source 42 to decrease the level of suction, for example, to the “sniff” level. The level of suction may be dynamically varied between zero, low, and high based on the concentration of the particulates as detected by the sensor 180. In the particulate detection mode, there may be no capacitive sensor, or the signals from the capacitive sensor may be weighted less in the determinations of the controller 40 relative to the signals from the optical sensor.

In an instrument operation mode, the vacuum source 42 is initially off or operated at a lower level. Based on the signals received from the sensor 102 indicative of a change in power delivered to the electrosurgical instrument 52, the controller 40 operates the vacuum source 42 to start, increase, decrease, or stop the level of suction. The controller 40 may analyze the received signals against a predetermined limit or according to a predefined algorithm. The signals may be provided to a trained machine learning model or one or more trained neural networks. For example, in response to the electrosurgical instrument 52 being activated or its power increased, the vacuum level is increased. In effect, the vacuum level is increased to accommodate a likely anticipated increase in surgical smoke based on the increased power being delivered to the electrosurgical instrument 52 being deployed at the surgical site.

The instrument operation mode may also utilize both of the sensors 102, 180. The controller 40 may adjust the vacuum level based on signals received from one of the sensors 102, 180, and further adjust the vacuum level based on the signals received from the other one of the sensor 102, 180. For example, the controller 40 decreases the vacuum level upon a detected deactivation of the electrosurgical instrument 52. However, residual surgical smoke may remain within the suction tube 60 and the filter assembly 30. After the initial decrease, the controller 40 may continue to operate the vacuum source 42 at the lower (e.g., non-zero) level either for a predetermined period, or until particulates detected by the sensor 180 no longer exceeds the predetermined threshold, after which the controller 40 may then terminate operation of the vacuum source 42. In effect, this ensures the suction tube 78 and the filter assembly 30 are cleared of any residual surgical smoke following deactivation of the electrosurgical instrument 52. Further, the use of multiple sensors provides redundancy, and allows the system to activate when smoke is generated through means other than electrosurgical instruments. The mode may be selected on the user interface of the display 46. Of course, the smoke evacuation unit 32 may be operated in a conventional “on” mode in which the vacuum source 42 is operated continuously at a desired level.

The modes may be customizable, i.e., utilizing aspects of the aformentioned modes based on inputted or sensed parameters of the surgical procedure. The modes may be customized based on type of procedure, duration of electrocautery within procedures or between procedures within the same surgical theater, duration of procedure, patient characteristics, number of medical personnel within the surgical theater, and the like, or combinations thereof. Therefore, the filter assembly 30 of the present disclosure advantageously provides for improved control of the vacuum source 42 of the smoke evacuation unit 32 in a manner requiring little setup from the user, and further in a manner that avoids inadvertent activation of the vacuum source 42 by other powered instruments and devices within the operating room. In particular, the user need only install the filter assembly 30 into the receptacle 36, and removably couple the suction tube 60 with the inlet ports 106 for the power cord 54 adjacent thereto, as after which the EM-based activation of the vacuum source 42 is readied for use.

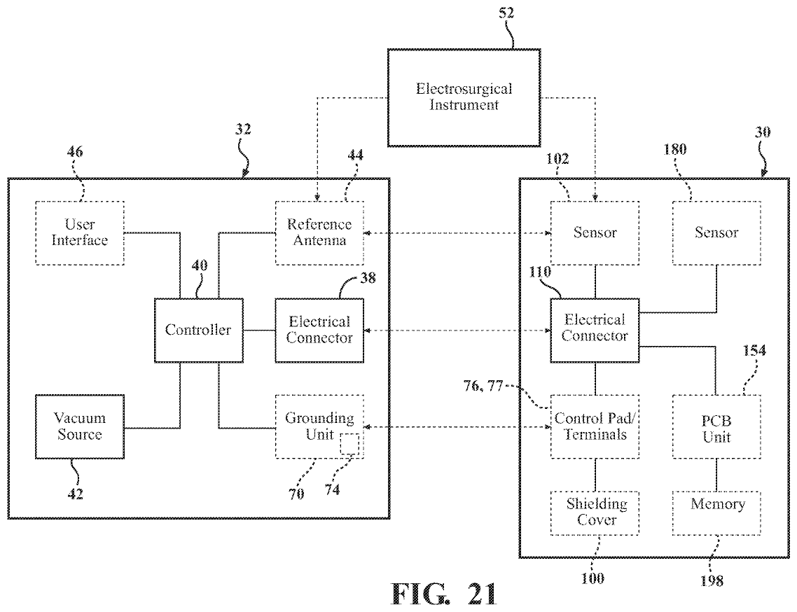

The grounding of the shielding layer 122 of the shielding cover 100, as discussed above—in addition to providing an EM barrier—is an exemplary means for ensuring authenticity or compatibility of the filter assembly 30 with the smoke evacuation unit 32. Further, it is implemented in a manner that is not prohibitively expensive to be implemented on a potentially disposable component. Th block diagram of FIG. 21 schematically represents various connections between the filter assembly 30, the smoke evacuation unit 32, the electrosurgical instrument 52, and certain subcomponents thereof. As described above, the smoke evacuation unit 32 includes the electronic connector 38, the controller 40, the vacuum source 42. Further, in some implementations, the smoke evacuation unit 32 may include the reference antenna 44, the display 46, and/or the grounding unit 70. The filter assembly 30 includes the electronic connector 110. The mating of the electronic connectors 38, 110 places the filter assembly 30 in electrical communication with the electronic connector 38 and the controller 40 of the smoke evacuation unit 32 to facilitate communication therebetween. In certain implementations, the filter assembly 30 includes the shielding cover 100, the contact pad(s) 76, the sensors 102,180, the PCB unit 154, and memory 198. In certain variants, one or more terminals 77 to be described in electrical communication with the electronic connector 110.

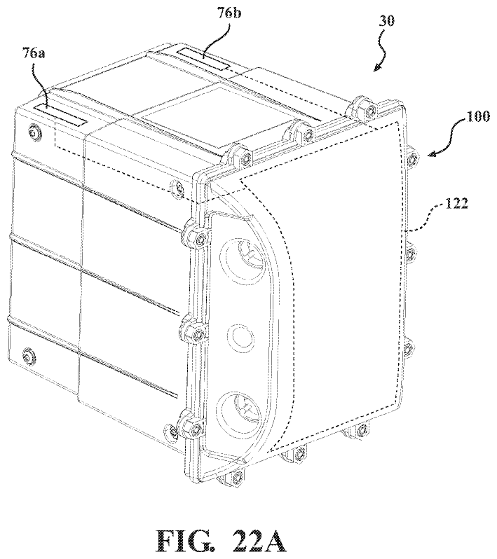

FIG. 22A depicts a variant of the first implementation of the filter assembly 30 in which there are two contact pads 76a, 76b in electrical communication with the shielding layer 122 of the shielding cover 100. For example, the contact pads 76a, 76b may be finger-like protrusions configured to engage the electrical terminals 74 of the smoke evacuation unit 32. FIG. 22B depicts another variant of the electrical pathway in which there are two terminals 77 in electrical communication with the shielding layer 122. The first and second terminals 77a, 77b may be disposed on or within the electronic connector 110 and arranged to engage corresponding terminals the electronic connector 38. For example, the first and second terminals 77a, 77b may be pins to engage receptacles of the electronic connector 38. The electrical communication may be established with wire leads or other suitable conductive arrangement.

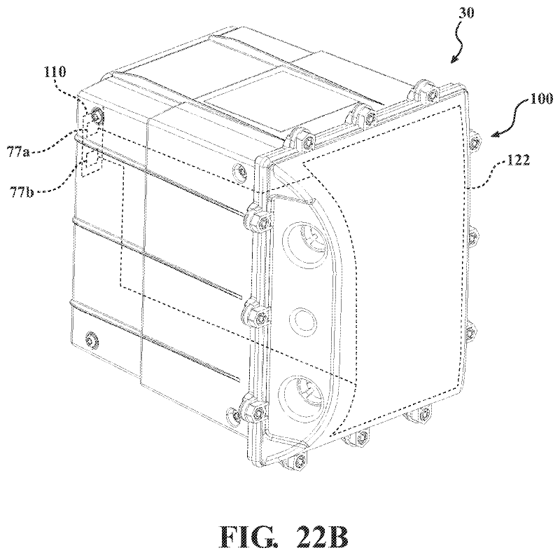

Referring to FIGS. 23A-25B, additional variants of electrical pathways between the smoke evacuation unit 32 and of the filter assembly 30 are schematically illustrated. Signals through the electrical pathways may be augmented, exploited, or otherwise utilized by the controller 40 to determine one or more electrical characteristics of the shielding cover 100, which is one manner by which the smoke evacuation unit 32 may determine the compatibility (e.g., authenticate) the filter assembly 30. In particular, an electrical signals from the controller 40 may be transmitted through the first terminal 77a and the shielding cover 100 to returned to the controller 40 through the second terminal 77b. In other words, the first terminal 77a, the first terminal 77b, and the conductive layer 122 of the shielding cover 100 form a circuit with the controller 40. The shielding cover 100 may have electrical characteristics which cause an electrical signal to be augmented as the signal passes through the shielding cover 100. For example, the shielding cover 100 may have an impedance, an inductance, a conductance, a resistance, and/or a capacitance which can be determined by the controller 40. Further, the shielding cover 100 may have a resonance frequency (e.g., of the material) associated with a predetermined voltage or current being transmitted through the circuit.

In order to confirm the authenticity or compatibility of the filter assembly 30, the controller 40 may transmit electrical signals to the shielding cover 100 of the filter assembly 30, and receive augmented electrical signals therefrom. The controller 40 may then compare the electrical signal transmitted to the shielding cover 100 against the augmented electrical signal received from the shielding cover 100 to determine one or more electrical characteristics described above. For example, and with reference to the variant of FIG. 23A, the controller 40 may set a first pin of the electronic connector 38 (which contacts a pin of the electronic connector 110) to a first voltage such that the first terminal 77a is set to the first voltage. The controller 40 may then permit electrical current to be drawn by the shielding cover 100 through the first terminal 77a. The electrical characteristics of the shielding cover 100 may cause a voltage drop between the first and second terminals 77a, 77b, and the controller 40 may detect a second voltage at a second pin of the electronic connector 38. If the voltage drop exceeds a predetermined threshold, the controller 40 determines that the electrical pathway has conductive material of at least similar properties (e.g., size, shape, material type) of that of a genuine filter assembly. In other words, the controller 40 may compare the augmented signal(s) against prestored signal characteristics to determine the authenticity of the filter assembly 30. Based thereon, the controller 40, in effect, may determine the filter assembly 30 includes the shielding cover 100, and authenticates the filter assembly 30 accordingly. In another variant, the controller 40 may compare the resistance of the shielding cover 100 to a predetermined resistance stored on the memory 198, which is accessible to the controller 40. If the filter assembly 30 is authenticated, the controller 40 permits the smoke evacuation unit 32 to operate as intended. If, however, a non-genuine filter assembly is not authenticated, the controller 40 may either prevent operation of the smoke evacuation unit 32, or disable one or more features (e.g., the instrument operation mode).

In certain implementations, the filter assembly 30 may include more than two terminals 77 to permit the controller 40 to measure the electrical characteristics of the shielding cover 100 along different pathways or vectors according to a predetermined authentication scheme. For example, the filter assembly 30 may include more than two terminals 77, and the controller may transmit an electrical signal to the shielding cover 100 through one of the terminals 77 and receive augmented signals via the remaining terminals 77. The electrical signal may be transmitted along different portions of the shielding cover 100 between the terminals 77. The controller 40 may determine electrical the characteristics of different portions of the shielding cover 100. Through material selection, dimensions, and the like, the different portions may be designed to have predefined electrical characteristics, and the controller 40 may require the measured electrical characteristics of each of the portions to be within an acceptable range to determine the filter assembly 30 is compatible.

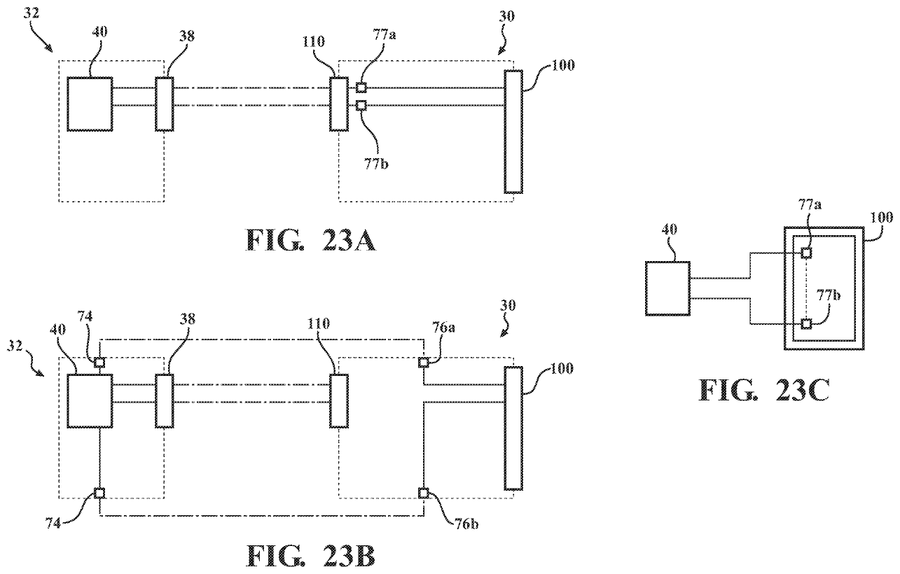

One such implementation is depicted in FIGS. 24A and 24B in which the filter assembly 30 which includes three terminals 77a, 77b, 77c in electrical communication with the shielding cover 100. The shielding layer 122 of the shielding cover 100 defines multiple electrical pathways configured to form alternative circuits with the controller 40 of the smoke evacuation unit 32 through which electrical communication is established between the controller 40 and the shielding cover 100. Each electrical pathway may have different electrical characteristics. For example, the controller 40 may be configured to transmit an electrical signal to the shielding cover 100 via the first terminal 77a and receive first and second augmented signals via the second and third terminals 77b, 77c, respectively. In this example, a first electrical signal conducted along the electrical pathway defined between the first and second terminals 77a, 77b may be altered according to the electrical characteristics of a first portion of the shielding cover 100. A first augmented signal is returned to the controller 40 via the second terminal 77b. The same or different electrical signal may be conducted along the electrical pathway defined between the first and third terminals 77a, 77c and may be altered according to the electrical characteristics of a second portion of the shielding cover 100. A second augmented signal is returned to the controller 40 via the first terminal 77a. The authentication schemes may include transmitting the signals across the terminals 77a, 77b, 77c in any order, grouping, and at any frequency. For example, two electrical signals may be transmitted to the shielding cover 100 through two of the three terminals 77, such as through the first and third terminals 77a, 77c, and at least one augmented signal may be received from the other one of the three terminals, such as the second terminal 77b. The controller 40 may compare the electrical signals to the augmented electrical signals, as affected by the electrical characteristics of portions of the shielding cover 100. The determined electrical characteristics may then be compared against prestored electrical characteristics to determine the authenticity of the filter assembly 30.

FIGS. 24C and 24D show another variant in which there are four terminals 77 77a, 77b, 77c, 77d. and the shielding layer 122 of the shielding cover 100 defining multiple electrical pathways configured to form alternative circuits. In this variant, the pairs of terminals 77 do not share a common terminal. Similar to the implementations discussed above, each electrical pathway may have different electrical characteristics, and may be measured according to the predefined calibration scheme of any timing, grouping, sequence, or the like. For example, the electrical signals could be transmitted to the first and third terminals 77a, 77c sequentially or simultaneously. Sequential transmission may result in the controller 40 receiving the augmented signals further according to a predetermined timing scheme, which also be used as an alternative to or in combination with the augmentation of the electrical signals for even more complex authentication schemes.

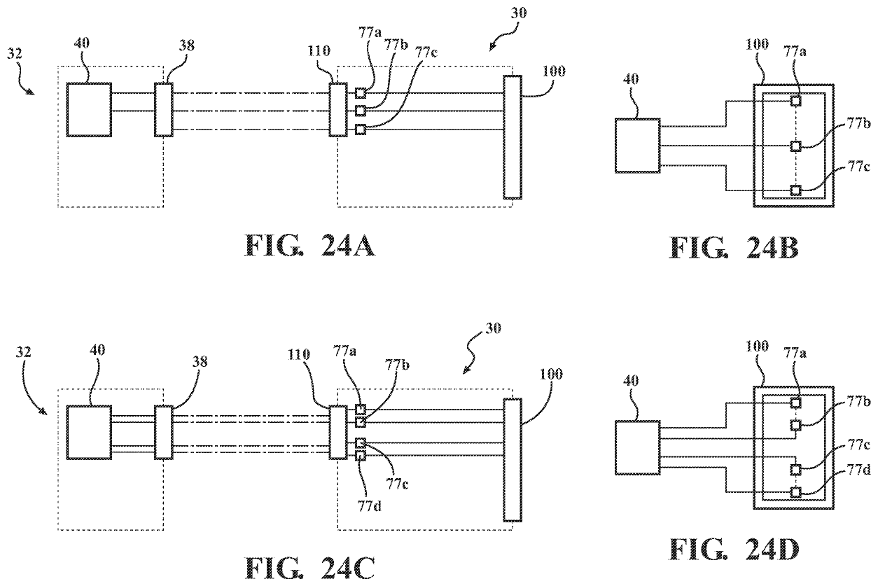

As discussed above, the shielding cover 100 of the filter assembly 30 may be grounded through the grounding unit 70 with the filter assembly 30 disposed in the receptacle 36. Another variant of the concept is schematically represented in FIGS. 25A and 25B in which the shielding cover 100 is configured to be arranged in electrical communication with a grounding element 195 of the smoke evacuation unit 32. The grounding element 195 may the same or different than the grounding unit 70. The shielding cover 100 may be in electrical communication with the grounding pin of the electronic connector 38, and the controller 40 determines the authenticity of the filter assembly 30 by confirming that the shielding cover 100 is grounded through the grounding element 195 (in addition to augmented signals received via the first or second terminals 77a, 77b).

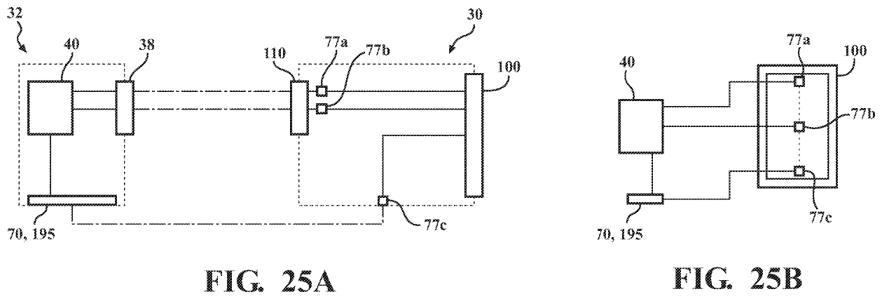

Referring now to FIG. 26, another variant of the electrical pathway is shown in which an electrical shorting element 214 extends between the first and second terminals 77a, 77b. The electrical shorting element 214 may be a jumper wire that returns an electrical signal. The first terminal 77a, the first terminal 77b, and the electrical shorting element 214 may define the electrical pathway configured to form a circuit from which the controller 40 may measure electrical characteristic(s). One example is the absence or presence of the electrical shorting element 214. The controller 40 may determine the authenticity of the filter assembly 30 by confirming that the first and second terminals 77a, 77b are shorted together. Another example includes determining whether the first and second terminals 77a, 77b and the electrical shorting element 214 are grounded to protective earth. Other electrical characteristics include impedance, inductance, conductance, and resistance, among others.

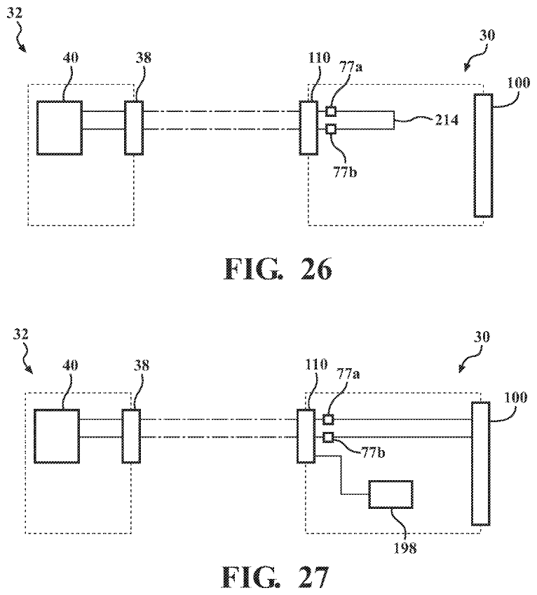

In certain implementations, the filter assembly 30 may include memory 198 storing calibration data, instructions, and/or other authentication-related data. The calibration data may be transmitted to and the controller 40 for processing. The controller 40 may authenticate the filter assembly 30 based on the data. Referring now to FIG. 27, the memory 198 is in electrical communication with the electronic connector 110. Through the electronic connector 110, and in particular the terminals 77a, 77b thereof, the memory 198 is arranged in electrical communication with the controller 40 of the smoke evacuation unit 32 with the filter assembly 30 disposed in the receptacle 36.

The controller 40 may be programmed or instructed to perform any one of more of the authentication schemes disclosed herein. The memory 198 may include non-transitory computer-readable medium storing instructions configured to be transmitted to the controller 40 to perform the authentication scheme(s). For example, the instructions from the memory 198 may instruct the controller 40 to determine whether the shielding cover 100 is grounded. For another example, the instructions from the memory 198 may cause the controller 40 to transmit an electrical signal through the electrical pathway(s). The controller 40 compares the augmented electrical signal against calibration data also received from the memory 198 to determine the authenticity of the filter assembly 30. The calibration data may be a model augmented signal and/or at least one predetermined electrical characteristic of the shielding cover 100.

In further optional implementations, the sensor 102 may be utilized to authenticate the filter assembly 30. With the sensor 102 being an electronic component, its absence or presence may be sensed or determined. In one variant, the smoke evacuation unit 32 includes a reference antenna 44 (see FIGS. 1, 2 and 21) in electronic communication with the controller 40. Once electronic communication is established via the electronic connectors 38, 110, the controller 40 may operate the reference antenna 44 to propagate an interrogation signal. The interrogation signal may be an electric field within a predefined frequency range. The sensor 102 is configured to receive the interrogation signal, which is returned to the controller 40 through the electronic connectors 38, 110. Based on a comparison between the interrogation signal and the returned signal, the controller 40 determines the presence of the sensor 102 to authenticate the filter assembly 30. For example, if no return signal is received, the controller 40 may determine that the filter assembly 30 is non-genuine. The sensor 102 may be selected or tuned to detect changes in the surrounding electric field only within a predefined frequency range such that multiple interrogation signals, each having a unique frequency, may be emitted by the reference antenna 44. The controller 40 may confirm the authenticity of the filter assembly 30 based on whether the sensor 102 responds to the interrogation signals appropriately, specifically whether the sensor 102 responds only to those within the predefined frequency range.

The sensor 102 may configured to measure a characteristic of the interrogation signal emitted by the reference antenna 44, and transmit the measured characteristic to the controller 40. The controller 40 may authenticate the filter assembly 30 based on a comparison between the interrogation signal and an operating parameter of the sensor 102. For example, the interrogation signal may be predefined and a complex signal with at least two frequency components, such as a first frequency component and a second frequency component. The operating parameter of the sensor 102 may be its resonant frequency, and the resonant frequency may be the same as the first frequency component of the interrogation signal. Therefore, when the interrogation signal is emitted by the reference antenna 44, the sensor 102 detects the first frequency component but not the second frequency component. The controller 40 receives the return signals from the sensor 102 indicative of detection of the first frequency component but not the second frequency component. The controller 40 determines that the filter assembly 30 is authentic. Conversely, should the controller 40 receive the return signals from the sensor 102 indicative of detection of both the first and second frequency components, the controller 40 may determine the filter assembly includes a non-genuine capacitive sensor, and therefore the filter assembly is non-genuine.

Certain optional implementations include the controller 40 comparing signals received from the sensor 102 to signals received from the reference antenna 44. The reference antenna 44 may include a receiver to detect electromagnetic energy, e.g., radiating from the power cord 54 of the electrosurgical instrument 52. The reference antenna 44 may propagate a predefined interrogation signal, which is then received at the reference antenna 44 after being augmented in the ambient environment. Likewise, the sensor 102 detects the interrogation signal. The controller 40 compares the received signals to determine the authenticity of the filter assembly 30. In first variant, the controller 40 may determine that the filter assembly 30 is authentic if the received signals are the same or similar. In other words, both the reference antenna 44 and the sensor 102 are detecting the same or similar altered electric fields. The variant may be well suited for implementations of the filter assembly 30 in which the sensor 102 is not shielded (i.e., via the barriers 62, 66 and the shielding cover 100), but rather exposed to electromagnetic energy present within the surrounding environment. In a second variant, the controller 40 may determine that the filter assembly 30 is authentic if the received signals are different. The variant may be well suited for implementations of the filter assembly 30 in which the sensor 102 is shielded from the ambient electric field. In effect, the controller 40 confirms that the sensor 102 is shielded by determining the sensor 102 is not detecting the interrogation signal as propagated by the reference antenna 44, and only any altered electric field from the power cord 54. A third variant includes the controller 40 comparing a measured change in the ambient electric field, as detected by the reference antenna 44, to the altered electric field through the inlet port 106, as detected by the sensor 102. The controller 40 authenticate the filter assembly 30 if the measured change is consistent with the altered electric field.

In certain optional implementations, the sensor 102 may be backdriven by the controller 40, such as through the electronic connectors 38, 110, and transmit a response to the controller 40. For example, the controller 40 may transmit an interrogation signal (i.e., apply an interrogation voltage) to the sensor 102 via one pin of the electronic connector 110, and receive an augmented signal via another pin of the electronic connector 110. The augmented signal can be used by the controller 40 to determine electrical characteristics of the sensor 102, and the controller 40 may authenticate the filter assembly 30 based on the electrical characteristics of the sensor 102 as indicated by the augmented signal. Since the signals are transmitted to and from the sensor 102 via the electronic connectors 38, 110, backdriving may be utilized to determine characteristics of the sensor in implementations in which the filter assembly 30 is shielded.

Calibration of the filter assembly 30 during manufacturing may include characteristics and/or operating parameter(s) of the sensor 102 being measured and stored on the memory 198 as programmed data. The programmed data may later be transmitted to the controller 40 to authenticate the filter assembly 30 on a filter-specific. For example, the smoke evacuation unit 32 interrogate the sensor 102 (e.g., via the reference antenna 44 or by backdriving), and the memory 198 may be configured to transmit the programmed data to the controller 40. The controller 40 may be configured to determine the authenticity of the filter assembly 30 according to the received, programmed data. For example, the programmed data may be indicative of the sensor 102 being configured to detect signals of a frequency range as determined during assembly and/or calibration, and the interrogation signal transmitted by the reference antenna 44 may propagate the signals within the frequency range. The controller 40 may compare frequency of the interrogation signal to the programmed data in order to confirm that the sensor 102 has detected signals of the specific frequency which match the programmed data. In other words, the controller 40 may determine the authenticity of the filter assembly 30 by confirming that the sensor 102 responds in a way which is consistent with the operating parameters measured during calibration and stored on the memory 198. The programmed data and/or calibration schemes may be the same between or unique to each of several models of the filter assembly, which may differ by size, capacity, filtration efficiency, features, lifespan, or the like.

Several embodiments have been discussed in the foregoing description. However, the implementations discussed herein are not intended to be exhaustive or limit the filter assembly to any particular form factor. The terminology which has been used is intended to be in the nature of words of description rather than of limitation. Many modifications and variations are possible in light of the above teachings and the system may be practiced otherwise than as specifically described. For example, in several implementations above, the methods are described in the context of transmitting and receiving signals associated with the electric field. However, it should be appreciated that similar means may be accomplished by using voltages across the pins of the electronic connectors 38, 110. The load between the pins enables the controller 40 to measure electrical characteristics of the shielding cover 100 and/or the sensor 102. Further, it is further contemplated that electromagnetic-based control of the vacuum source 42 with the filter assembly 30 of the present disclosure may be utilized with other types of powered surgical devices that include a power cord.

Additional inventive aspects of the present disclosure are made with reference to the following exemplary clauses:
Clause 1—A filter assembly for a smoke evacuation unit that includes a receptacle, an electronic connector, and a controller in electrical communication with the electronic connector, the filter assembly including: a housing defining a suction outlet and one or more inlet ports; a filter disposed within the housing; an electronic connector configured to be arranged in electrical communication with the electronic connector of the smoke evacuation unit with the filter assembly disposed in the receptacle; a first terminal in electrical communication with the electronic connector; a second terminal in electrical communication with the electronic connector; and a shielding cover coupled to the housing, wherein at least a portion of the shielded cover includes conductive material configured to provide an electromagnetic barrier, and wherein the shielding cover is in electrical communication with the first and second terminals.
Clause 2—The filter assembly of clause 1, wherein the first terminal and the second terminal are disposed on or within the electronic connector.
Clause 3—The filter assembly of clause 2, wherein the electronic connector of the filter assembly is a plug, and wherein the first and second terminals are pins of the plug.
Clause 4—The filter assembly of clause 1, wherein the first terminal and the second terminal are conductive members coupled to the housing of the filter assembly, and wherein the first terminal and the second terminal are configured to engage terminals of the smoke evacuation unit.
Clause 5—The filter assembly of clause 4, wherein the conductive members are conductive fingers extending from the housing.
Clause 6—The filter assembly of any one of clauses 1-5, wherein the controller is configured to transmit a signal to the shielding cover, and receive an augmented electrical signal that is augmented based on at least one electrical characteristic of the shielding cover.
Clause 7—The filter assembly of clause 6, further including a memory unit storing calibration data corresponding to a model augmented electrical signal or at least one electrical characteristic of the cover, wherein the memory unit is configured to transmit the calibration data to the controller for comparison against the augmented electrical signal.
Clause 8—The filter assembly of any one of clauses 1-5, wherein the first terminal, the second terminal, and the shielding cover define an electrical pathway configured to form a circuit with the controller of the smoke evacuation unit for at least one electrical characteristic of the circuit to be determined by the controller to authenticate the filter assembly.
Clause 9—The filter assembly of clause 8, wherein the electrical pathway is ungrounded with the electronic connector not removably coupled to the electronic connector of the smoke evacuation unit, and wherein the electrical pathway is configured to be grounded through the electronic connector being removably coupled to the electronic connector.
Clause 10—The filter assembly of clause 9, further including a ground terminal in electronic communication with the shielding cover, wherein the shielding cover is arranged to be in electrical communication with a grounding element of the smoke evacuation unit with the filter assembly disposed in the receptacle.
Clause 11—The filter assembly of clause 10, wherein the ground terminal is disposed on the electronic connector.
Clause 12—The filter assembly of any one of clauses 6-11, wherein the at least one electrical characteristic is one of impedance, voltage, resistance, and current transmitted through the circuit at a resonance frequency of the shielding cover.
Clause 13—The filter assembly of clause 12, wherein the at least one electrical characteristic is a resonance frequency of the shielding cover associated with a predetermined voltage or current being transmitted through the circuit.
Clause 14—The filter assembly of any one of clauses 1-13, further including a third terminal in electrical communication with the shielding cover, wherein the third terminal and each of the first and second terminals are configured to form alternative circuits associated with a different electrical characteristic.
Clause 15—The filter assembly of clause 14, wherein the controller is configured to determining the electrical characteristics of the circuit and the alternative circuits across different pathways according to a predetermined calibration scheme.
Clause 16—The filter assembly of clause 15, wherein the predetermined calibration scheme includes determining the electrical characteristic of the circuit and the alternative circuits in a predefined sequence.
Clause 17—The filter assembly of clause 15, wherein the predetermined calibration scheme includes determining the electrical characteristic across of the circuit and the alternative circuits simultaneously.
Clause 18—The filter assembly of any one of clauses 1-17, further including a capacitive sensor disposed within the housing and in electronic communication with the electronic connector, wherein the capacitive sensor is configured to detect changes in the electric field.
Clause 19—The filter assembly of clause 18, wherein the capacitive sensor is configured to detect changes in the electric field within a predefined frequency range, and wherein the capacitive sensor is configured to receive an interrogation signal within the predefined frequency range, and wherein the controller to determine presence of the capacitive sensor based on a received signal from the capacitive sensor in response to the interrogation signal.
Clause 20—A filter assembly for a smoke evacuation unit that includes a receptacle, an electronic connector, and a controller in electrical communication with the electronic connector, the filter assembly including: a housing defining a suction outlet and one or more inlet ports; a filter disposed within the housing; an electronic connector configured to be in electrical communication with the electronic connector of the smoke evacuation unit with the filter assembly disposed in the receptacle; a first terminal in electrical communication with the electronic connector; a second terminal in electrical communication with the electronic connector; an electrical short extending between the first and second terminals, wherein the electrical short is configured to be arranged in electrical communication with the controller of the smoke evacuation unit with the filter assembly disposed in the receptacle.
Clause 21—The filter assembly of clause 20, wherein the first terminal and the second terminal are disposed on or within the electronic connector.
Clause 22—The filter assembly of clause 20 or 21, wherein the first terminal and the second terminal are wire leads, and wherein the electrical short is a lead extending between the wire leads.
Clause 23—The filter assembly of any one of clauses 20-22, wherein the first terminal, the second terminal, and the electrical short define an electrical pathway configured to form a circuit, and wherein the controller is configured to determine at least one electrical characteristic of the circuit.
Clause 24—The filter assembly of clause 23, wherein the electrical pathway is ungrounded with the electronic connector of the filter assembly not removably coupled to the electronic connector of the smoke evacuation unit, and wherein the electrical pathway is configured to be grounded through the electronic connector being removably coupled to the electronic connector.
Clause 25—The filter assembly of clause 23, wherein the electrical characteristic is whether the circuit is grounded to protective Earth.
Clause 26—The filter assembly of any one of clauses 20-25, wherein the at least one electrical characteristic is one of impedance, voltage, resistance, and current transmitted through the circuit.
Clause 27—A filter assembly for a smoke evacuation unit that includes a receptacle, an electronic connector, and a controller, the filter assembly including: a housing configured to be removably inserted into the receptacle, wherein the housing defines a suction outlet and one or more inlet ports; a filter disposed within the housing; an electronic connector configured to be coupled with the electronic connector of the smoke evacuation unit; a capacitive sensor disposed within the housing and in electronic communication with the electronic connector, wherein the capacitive sensor is configured to operate at one or more operating parameters and be arranged in electromagnetic communication with a reference antenna of the smoke evacuation unit, wherein the capacitive sensor is configured to detect an interrogation signal emitted by the reference antenna, and transmit the measured characteristic to the controller for the controller to authenticate the filter assembly.
Clause 28—A filter assembly for a smoke evacuation unit that includes a receptacle, an electronic connector, and a controller, the filter assembly including: a housing configured to be removably inserted into the receptacle, wherein the housing defines a suction outlet and one or more inlet ports; a filter disposed within the housing; an electronic connector configured to be in electrical communication with the electronic connector of the smoke evacuation unit; a capacitive sensor disposed within the housing and in electrical communication with the electronic connector, wherein the capacitive sensor is includes one or more electrical characteristics that is measured during assembly or calibration of the filter assembly; and memory in electrical communication with the electronic connector and storing programmed data including the electrical characteristics of the capacitive sensor, wherein the capacitive sensor is configured to receive an interrogation signal from the smoke evacuation unit and the memory unit is configured to transmit the programmed data to the controller in response to the capacitive sensor receiving the interrogation signal.
Clause 29—A filter assembly for a smoke evacuation unit that includes a receptacle, an electronic connector, and a controller, the filter assembly including: a housing configured to be removably inserted into the receptacle, wherein the housing defines a suction outlet and one or more inlet ports; a filter disposed within the housing; an electronic connector configured to be in electrical communication with the electronic connector of the smoke evacuation unit; and a capacitive sensor disposed within the housing and in electrical communication with the electronic connector, wherein the capacitive sensor is configured to be backdriven by an interrogation signal transmitted from the controller via the electronic connector.
Clause 30—A system including: a smoke evacuation unit including a vacuum source, a shielded receptacle, a reference antenna, a controller in electronic communication with the reference antenna, wherein the reference antenna is positioned to measure changes in ambient electric field; a filter assembly configured to be removably inserted into the shielded receptacle to be in fluid communication with the vacuum source, wherein the filter assembly includes: a housing including a shielded cover defining one or more inlet ports; a filter disposed within the housing; a capacitive sensor disposed within the housing so as to be electromagnetically shielded from the surgical environment by the shielding cover and the shielded receptacle, wherein the capacitive sensor is configured to measure changes in an altered electric field through the one or more inlet ports, wherein the controller is configured to authenticate the filter assembly on the measured changes of the ambient electric field from the reference antenna and the measured changes of the altered electric field from the capacitive sensor of the filter assembly.
Clause 31—A system including: a smoke evacuation unit including a vacuum source, a shielded receptacle, a reference antenna positioned within the shielded receptacle, a controller in electronic communication with the reference antenna; a filter assembly configured to be removably inserted into the shielded receptacle to be in fluid communication with the vacuum source, wherein the filter assembly includes: a housing including a shielding cover defining one or more inlet ports; a filter disposed within the housing; and a capacitive sensor disposed within the housing, wherein the capacitive sensor and the reference antenna are positioned in electromagnetic communication with one another with the filter assembly removably positioned within the shielded receptacle, and electromagnetically shielded from the surgical environment.
Clause 32—A method of operating a smoke evacuation unit including a receptacle, an electronic connector, and a controller, the method including: detecting, through an electronic connection, that a filter assembly has been inserted into the receptacle; determining, with the controller and through the electronic connection, an electrical characteristic of a shielded cover of the filter assembly; and authenticating, with the controller the filter assembly with the smoke evacuation unit based on the electrical characteristic.
Clause 33—The method of clause 32, further including: receiving, from memory of the filter assembly, calibration data corresponding to a model augmented electrical signal the shielded cover; transmitting, through the electronic connection, an interrogation signal to the filter assembly; comparing the measured electrical characteristic against the augmented electrical signal from the interrogation signal; and determining compatibility of the filter assembly with the smoke evacuation unit based on the comparison.
Clause 34—A method of operating a smoke evacuation unit including a receptacle, an electronic connector, and a controller, the method including: detecting, through an electronic connection, that a filter assembly has been inserted into the receptacle; determining, with the controller and through the electronic connection, an electrical characteristic of an electrical pathway; and authenticating, with the controller, the filter assembly with the smoke evacuation unit based on the electrical characteristic.
Clause 35—A method of operating a smoke evacuation unit including a receptacle, an electronic connector, and a controller, wherein a filter assembly is configured to be removably inserted into the receptacle and including a capacitive sensor, the method including: detecting, with the capacitive sensor, changes in electric field being through one or more inlet ports to which a suction tube is removably coupled; receiving, at the controller and through the electronic connector, signals from the capacitive sensor; and controlling, with the controller, operation of the vacuum source based on the signals.
Clause 36—The method of clause 35, wherein the filter assembly further includes a optical sensor, the method further including: detecting, with the optical sensor, particulates in surgical smoke; receiving, at the controller and through the electronic connector, additional signals from the optical sensor; and controlling, with the controller, the vacuum source based on the signals and the additional signals.
Clause 37—The method of clause 35 or 36, wherein the smoke evacuation unit comprises a grounding unit including one or more terminals, and wherein the filter assembly comprises a contact pad, the method further including: determine a presence of an electrical pathway between the contact pad and the one or more terminals of the grounding unit; and controlling operation of the vacuum source based on a determined presence of the electrical pathway.

1. A filter assembly for a smoke evacuation unit, the filter assembly comprising:
a housing defining a suction outlet, and one or more inlet ports configured to receive a suction tube;
a filter disposed within the housing; and
a capacitive sensor disposed within the housing and configured to detect changes in electric field through the one or more inlet ports.

2. (canceled)

3. The filter assembly of claim 1, wherein the housing comprises a port panel defining the one or more inlet ports, and wherein the sensor is disposed adjacent to an inner side of the port panel.

4. The filter assembly of claim 3, wherein the housing further comprises a partition spaced apart from the inner side of the port panel to define a sensor compartment therebetween, and wherein the sensor is disposed within the sensor compartment.

5. The filter assembly of claim 4, wherein the housing further comprises one or more coupling features disposed within the sensor compartment and configured to support the sensor.

6. The filter assembly of claim 4, wherein the one or more inlet ports extend through the partition to bypass the sensor compartment.

7. The filter assembly of claim 1, wherein the sensor comprises a curvilinear edge contoured to at least one of the one or more inlet ports.

8. The filter assembly of claim 1, wherein the one or more inlet ports comprises an upper inlet port, a middle inlet port, and a lower inlet port, and wherein the sensor comprises an upper portion disposed between the upper and middle inlet ports, and a lower portion disposed between the middle and lower inlet ports.

9-15. (canceled)

16. The filter assembly of claim 1, wherein the housing further comprises a filter casing, and a shielding cover coupled to the filter casing, and wherein the shielding cover comprises conductive material configured to provide an electromagnetic barrier.

17. The filter assembly of claim 16, wherein the shielding cover further comprises an inner casing, an outer casing, and a conductive layer of the conductive material disposed between the outer casing and the inner casing.

18. The filter assembly of claim 16, further comprising a contact pad coupled to the housing and configured to engage one or more terminals of the smoke evacuation unit, wherein the shielding cover is in electrical communication with the contact pad.

19. The filter assembly of claim 18, further comprising a conductive bridge directly contacting each of the contact pad and the shielding cover so as to provide electrical communication therebetween.

20. The filter assembly of claim 18, wherein a side of the housing comprises a step defining an upper portion that is recessed from a lower portion, and wherein the contact pad is disposed on the upper portion of the side.

21. The filter assembly of claim 20, wherein the upper portion of the side defines one or more windows through which the contact pad is exposed.

22. The filter assembly of claim 21, wherein the upper portion of the side comprises a ridge adjacent to the one or more windows and configured to provide defeatable retention with the smoke evacuation unit.

23. A filter assembly for a smoke evacuation unit, the filter assembly comprising:
a housing defining a suction outlet, and one or more inlet ports configured to receive a suction tube; and
a filter disposed within the housing,
wherein the housing comprises a shielding cover comprising conductive material configured to provide an electromagnetic barrier; and
a sensor coupled to the shielding cover and configured to detect changes in electric field through the one or more inlet ports.

24. (canceled)

25. The filter assembly of claim 23, wherein the shielding cover further comprises an inner casing, an outer casing, and a conductive layer of the conductive material disposed between the outer casing and the inner casing.

26. The filter assembly of claim 25, further comprising a contact pad configured to engage one or more terminals of the smoke evacuation unit, wherein the conductive layer of the shielding cover is in electrical communication with the contact pad.

27. The filter assembly of claim 26, further comprising a conductive bridge contacting each of the contact pad and the conductive layer so as to provide electrical communication therebetween.

28-40. (canceled)

41. The filter assembly of claim 1, further comprising a liquid separator coupled to the housing and configured to direct surgical smoke about a lower edge of a barrier such that momentum causes liquid entrained with the surgical smoke to descend into a liquid collection compartment.

42. The filter assembly of claim 1, further comprising one or more port covers selectively disposed over a respective one of the one or more inlet ports, and wherein the one or more port covers comprise conductive material configured to provide an electromagnetic barrier.

43. The filter assembly of claim 1, wherein the housing defines a cavity sized to form a handle for grasping the filter assembly, and wherein the handle is on a side of the housing opposite the one or more inlet ports.

44-62. (canceled)

63. A filter assembly for a smoke evacuation unit, the filter assembly comprising:
a housing defining a suction outlet, and comprising a port panel defining one or more inlet ports configured to receive a suction tube, wherein the housing further comprises a partition spaced apart from the inner side of the port panel to define a sensor compartment therebetween;
a filter disposed within the housing; and
a sensor disposed within the sensor compartment and adjacent to an inner side of the port panel, wherein the sensor is configured to detect external operation of a powered surgical device.
